(19) United States
(12) Patent Application Publication
(10) Pub. No.: US 2012/0220665 A1
(43) Pub. Date: Aug. 30, 2012

(54) Inhibiting Neurotransmitter Reuptake

(75) Inventors: Elliott Richelson, Ponte Vedra Beach, FL (US); Abdul H. Fauq, Jacksonville, FL (US); Dola Sinha, Jacksonville, FL (US)

(73) Assignee: MAYO FOUNDATION FOR MEDICAL EDUCATION AND RESEARCH, Rochester, MN (US)

(21) Appl. No.: 13/505,899

(22) PCT Filed: Nov. 2, 2010
(86) PCT No.: PCT/US10/55065

§ 371 Date: May 3, 2012

Related U.S. Application Data
(60) Provisional application No. 61/257,744, filed on Nov. 3, 2009.

(51) Int. Cl. A61K 31/135; C07C 215/30

(52) U.S. Cl. 514/652; 514/653; 564/346; 564/355

(57) ABSTRACT

This document relates to compounds as well as methods and materials involved in modulating neurotransmitter reuptake. For example, compounds, methods for synthesizing compounds, and methods for inhibiting neurotransmitter reuptake are provided.

### CROSS-REFERENCE TO RELATED APPLICATIONS

This application claims priority to U.S. Provisional Application Ser. No. 61/257,744, filed on Nov. 3, 2009. The disclosures of the prior application is considered part of (and are incorporated by reference in) the disclosure of this application.

### BACKGROUND

1. Technical Field

This document relates to compounds as well as methods and materials involved in modulating neurotransmitter reuptake.

2. Background Information

Neuronal signals are transmitted from cell to cell at specialized sites of contact known as synapses. The usual mechanism of transmission is indirect. The cells are electrically isolated from one another, the pre-synaptic cell being separated from the postsynaptic cell by a narrow synaptic cleft. A change of electrical potential in the pre-synaptic cell triggers it to release signaling molecules known as neurotransmitters. The neurotransmitters rapidly diffuse across the synaptic cleft and provoke an electrical change in the postsynaptic cell by binding to neurotransmitter receptorgated ion channels. After release, the excess neurotransmitters are rapidly removed, either by specific enzymes in the synaptic cleft or by reuptake into the pre-synaptic cell or surrounding glial cells. Reuptake is mediated by a variety of neurotransmitter transporters. Rapid removal ensures both spatial and temporal precision of signaling at a synapse. For example, rapid reuptake can prevent excess neurotransmitters from influencing neighboring cells and can clear the synaptic cleft before the next pulse of neurotransmitter release so that the timing of repeated, rapid signaling events is accurately communicated to the postsynaptic cell.

An imbalance of neurotransmitters in the brain can occur when not enough neurotransmitter is made and released from pre-synaptic cells or the reuptake of neurotransmitters by pre-synaptic cells is too rapid. If neurotransmitters such as serotonin, norepinephrine, or dopamine are not made and released in effective amounts or are cleared from the synaptic cleft too quickly, then cell-to-cell communication can be affected. Clinical manifestations of such imbalances include depression and anxiety disorders. Serotonin-, norepinephrine-, dopamine-reuptake inhibitors (SNDRIs) represent a class of potent, wide-spectrum antidepressant medications that inhibit the reuptake of these neurotransmitters back into pre-synaptic cells. Inhibiting neurotransmitter reuptake can increase the amount of neurotransmitter present in the synapse, thus helping to normalize the transmission of neuronal signals and alleviate the symptoms of depression and anxiety disorders.

### SUMMARY

This document relates to compounds as well as methods and materials involved in modulating neurotransmitter reuptake. For example, this document provides compounds (e g, amine compounds), methods for synthesizing compounds (e.g., amine compounds), and methods for inhibiting neurotransmitter reuptake. The compounds provided herein can be used as potent, wide-spectrum antidepressant medications for inhibiting neurotransmitter reuptake and treating anxiety or depressive disorders. In some cases, a compound provided herein can be used to treat pain. In addition, the methods provided herein for synthesizing compounds allow for synthesis in a reliable and efficient manner.

In general, one aspect of this document features a composition comprising, or consisting essentially of, 1-(4-fluorophenyl)-3-(methylamino)-2-(naphthalen-2-yl)propan-1-ol or a salt thereof The 1-(4-fluorophenyl)-3-(methylamino)-2-(naphthalen-2-yl)propan-1-ol can comprise, or can consist of, (1R, 2R)-1-(4-fluorophenyl)-3-(methylamino)-2-(naphthalen-2-yl)propan-1-ol or (1S, 2S)-1-(4-fluorophenyl)-3-(methylamino)-2-(naphthalen-2-yl)propan-1-ol. The 1-(4-fluorophenyl)-3-(methylamino)-2-(naphthalen-2-yl)propan-1-ol can comprise, or can consist of, (1R, 2S)-1-(4-fluorophenyl)-3-(methylamino)-2-(naphthalen-2-yl)propan-1-ol or (1S, 2R)-1-(4-fluorophenyl)-3-(methylamino)-2-(naphthalen-2-yl)propan-1-ol. The 1-(4-fluorophenyl)-3-(methylamino)-2-(naphthalen-2-yl)propan-1-ol can comprise, or can consist of: (i) two compounds selected from the group consisting of (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2 S)-1-(4-fluorophenyl)-3-(methylamino)-2-(naphthalen-2-yl)propan-1-ol, and (1S, 2R)-1-(4-fluorophenyl)-3-(methylamino)-2-(naphthalen-2-yl)propan-1-ol; or (ii) three compounds selected from the group consisting of (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2S)-1-(4-fluorophenyl)-3-(methylamino)-2-(naphthalen-2-yl)propan-1-ol, and (1S, 2R)-1-(4-fluorophenyl)-3-(methylamino)-2-(naphthalen-2-yl)propan-1-ol; or (iii) (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2S)-1-(4-fluorophenyl)-3-(methylamino)-2-(naphthalen-2-yl)propan-1-ol, and (1S, 2R)-1-(4-fluorophenyl)-3-(methylamino)-2-(naphthalen-2-yl)propan-1-ol. The composition can comprise a pharmaceutically acceptable carrier.

In another embodiment, this document features a composition comprising, or consisting essentially of, 1-(4-bromophenyl)-3-(methylamino)-2-phenylpropan-1-ol or a salt thereof. The 1-(4-bromophenyl)-3-(methylamino)-2-phenylpropan-1-ol can comprise, or can consist of, (1R, 2R)-1-(4-bromophenyl)-3-(methylamino)-2-phenylpropan-1-ol or (1S, 2S)-1-(4-bromophenyl)-3-(methylamino)-2-phenylpropan-1-ol. The 1-(4-bromophenyl)-3-(methylamino)-2-phenylpropan-1-ol can comprise, or can consist of, (1R, 2S)-1-(4-bromophenyl)-3-(methylamino)-2-phenylpropan-1-ol or (1S, 2R)-1-(4-bromophenyl)-3-(methylamino)-2-phenylpropan-1-ol. The 1-(4-bromophenyl)-3-(methylamino)-2-phenylpropan-1-ol can comprise, or can consist of: (i) two compounds selected from the group consisting of (1R, 2R)-1-(4-bromophenyl)-3-(methylamino)-2-phenylpropan-1-ol, (1S, 2S)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol; or (ii) three compounds selected from the group consisting of (1R,2R)-1-(4-bromophenyl)-3-(methylamino)-2-phenylpropan-1-ol, (1S, 2S)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol; or (iii) (1R, 2R)-1-(4-bromophenyl)-3-(methylamino)-2-phenylpropan-1-ol, (1S, 2S)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol. The composition can comprise a pharmaceutically acceptable carrier.

In another embodiment, this document features a composition comprising, or consisting essentially of, 3,3′-(heptane-1,7-diylbis(oxy))bis(N-methyl-2-(napthalen-2-yl)-3-phenylpropan-1-amine) or a salt thereof. The 3,3′-(heptane-1,7-diylbis(oxy))bis(N-methyl-2-(naphthalen-2-yl)-3-phenylpropan-1-amine) can comprise, or can consist of, (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine). The 3,3′-(heptane-1,7-diylbis(oxy))bis(N-methyl-2-(naphthalen-2-yl)-3-phenylpropan-1-amine) can comprise, or can consist of, (2S, 2′S, 3R, 3′R)-3,3′-(heptane-1,7-diylbis(oxy))bis(N-methyl-2-(naphthalen-2-yl)-3-phenylpropan-1-amine) or (2R, 2′R, 3S, 3′S)-3,3′-(heptane-1,7-diylbis(oxy))bis(N-methyl-2-(naphthalen-2-yl)-3-phenylpropan-1-amine) The 3-azido-N-methyl-2-(naphthalen-2-yl)-3-phenylpropan-1-amine can comprise, or can consist of: (i) two compounds selected from the group consisting of (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine); or (ii) three compounds selected from the group consisting of (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine); or (iii) (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine). The composition can comprise a pharmaceutically acceptable carrier.

In another embodiment, this document features a composition comprising, or consisting essentially of, 1-(4-fluorophenyl)-3-(methylamino)-2-phenylpropan-1-ol or a salt thereof. The 1-(4-fluorophenyl)-3-(methylamino)-2-phenylpropan-1-ol can comprise, or can consist of, (1R, 2R)-1-(4-fluorophenyl)-3-(methylamino)-2-phenylpropan-1-ol or (1S, 2S)-1-(4-fluorophenyl)-3-(methylamino)-2-phenylpropan-1-ol. The 1-(4-fluorophenyl)-3-(methylamino)-2-phenylpropan-1-ol can comprise, or can consist of, (1R, 2S)-1-(4-fluorophenyl)-3-(methylamino)-2-phenylpropan-1-ol or (1S, 2R)-1-(4-fluorophenyl)-3-(methylamino)-2-phenylpropan-1-ol. The 1-(4-fluorophenyl)-3-(methylamino)-2-phenylpropan-1-ol can comprise, or can consist of: (i) two compounds selected from the group consisting of (1R, 2R)-1-(4-fluorophenyl)-3-(methylamino)-2-phenylpropan-1-ol, (1S, 2S)-1-(4-fluorophenyl)-3-(methylamino)-2-phenylpropan-1-ol, (1R, 2S)-1-(4-fluorophenyl)-3-(methylamino)-2-phenylpropan-1-ol, and (1S, 2R)-1-(4-fluorophenyl)-3-(methylamino)-2-phenylpropan-1-ol; or (ii) three compounds selected from the group consisting of (1R, 2R)-1-(4-fluorophenyl)-3-(methylamino)-2-phenylpropan-1-ol, (1S, 2S)-1-(4-fluorophenyl)-3-(methylamino)-2-phenylpropan-1-ol, (1R, 2S)-1-(4-fluorophenyl)-3-(methylamino)-2-phenylpropan-1-ol, and (1S, 2R)-1-(4-fluorophenyl)-3-(methylamino)-2-phenylpropan-1-ol; or (iii) (1R, 2R)-1-(4-fluorophenyl)-3-(methylamino)-2-phenylpropan-1-ol, (1S, 2S)-1-(4-fluorophenyl)-3-(methylamino)-2-phenylpropan-1-ol, (1R, 2S)-1-(4-fluorophenyl)-3-(methylamino)-2-phenylpropan-1-ol, and (1S, 2R)-1-(4-fluorophenyl)-3-(methylamino)-2-phenylpropan-1-ol. The composition can comprise a pharmaceutically acceptable carrier.

In another embodiment, this document features a composition comprising, or consisting essentially of, 3-(methylamino)-2-(naphthalen-2-yl)-1-phenylpropyl acetate or a salt thereof. The 3-(methylamino)-1-(naphthalen-2-yl)-2-phenylpropan-1-ol can comprise, or can consist of, (1R, 2R)-3-(methylamino)-1-(naphthalen-2-yl)-2-phenylpropan-1-ol or (1S, 2R)-3-(methylamino)-1-(naphthalen-2-yl)-2-phenylpropan-1-ol. The 3-(methylamino)-1-(naphthalen-2-yl)-2-phenylpropan-1-ol can comprise, or can consist of, (1R, 2S)-3-(methylamino)-1-(naphthalen-2-yl)-2-phenylpropan-1-ol or (1S, 2R)-3-(methylamino)-1-(naphthalen-2-yl)-2-phenylpropan-1-ol. The 3-(methylamino)-1-(naphthalen-2-yl)-2-phenylpropan-1-ol can comprise, or can consist of: (i) two compounds selected from the group consisting of (1R, 2R)-3-(methylamino)-1-(naphthalen-2-yl)-2-phenylpropan-1-ol, (1S, 2S)-3-(methylamino)-1-(naphthalen-2-yl)-2-phenylpropan-1-ol, (1R, 2S)-3-(methylamino)-1-(naphthalen-2-yl)-2-phenylpropan-1-ol, and (1S, 2R)-3-(methylamino)-1-(naphthalen-2-yl)-2-phenylpropan-1-ol; or (ii) three compounds selected from the group consisting of (1R, 2R)-3-(methylamino)-1-(naphthalen-2-yl)-2-phenylpropan-1-ol, (1S, 2S)-3-(methylamino)-1-(naphthalen-2-yl)-2-phenylpropan-1-ol, (1R, 2S)-3-(methylamino)-1-(naphthalen-2-yl)-2-phenylpropan-1-ol, and (1S, 2R)-3-(methylamino)-1-(naphthalen-2-yl)-2-phenylpropan-1-ol; or (iii) (1R, 2R)-3-(methylamino)-1-(naphthalen-2-yl)-2-phenylpropan-1-ol, (1S, 2S)-3-(methylamino)-1-(naphthalen-2-yl)-2-phenylpropan-1-ol, (1R, 2S)-3-(methylamino)-1-(naphthalen-2-yl)-2-phenylpropan-1-ol, and (1S, 2R)-3-(methylamino)-1-(naphthalen-2-yl)-2-phenylpropan-1-ol. The composition can comprise a pharmaceutically acceptable carrier.

In another embodiment, this document features a composition comprising, or consisting essentially of, 3-chloro-N,N-dimethyl-2-(naphthalen-2-yl)-3-phenylpropan-1-amine or a salt thereof The 3-chloro-N,N-dimethyl-2-(naphthalen-2-yl)-3-phenylpropan-1-amine can comprise, or can consist of, (2R, 3R)-3-chloro-N,N-dimethyl-2-(naphthalen-2-yl)-3-phenylpropan-1-amine or (2S, 3S)-3-chloro-N,N-dimethyl-2-(naphthalen-2-yl)-3-phenylpropan-1-amine The 3-chloro-N,N-dimethyl-2-(naphthalen-2-yl)-3-phenylpropan-1-amine can comprise, or can consist of, (2R, 3S)-3-chloro-N,N-dimethyl-2-(naphthalen-2-yl)-3-phenylpropan-1-amine or (2S, 3R)-3-chloro-N,N-dimethyl-2-(naphthalen-2-yl)-3-phenylpropan-1-amine The 3-chloro-N,N-dimethyl-2-(naphthalen-2-yl)-3-phenylpropan-1-amine can comprise, or can consist of: (i) two compounds selected from the group consisting of (2R, 3R)-3-chloro-N,N-dimethyl-2-(naphthalen-2-yl)-3-phenylpropan-1-amine, (2S, 3 S)-3-chloro-N,N-dimethyl-2-(naphthalen-2-yl)-3-phenylpropan-1-amine, (2R, 3 S)-3-chloro-N,N-dimethyl-2-(naphthalen-2-yl)-3-phenylpropan-1-amine, and (2S, 3R)-3-chloro-N,N-dimethyl-2-(naphthalen-2-yl)-3-phenylpropan-1-amine; or (ii) three compounds selected from the group consisting of (2R, 3R)-3-chloro-N,N-dimethyl-2-(naphthalen-2-yl)-3-phenylpropan-1-amine, (2S, 3 S)-3-chloro-N,N-dimethyl-2-(naphthalen-2-yl)-3-phenylpropan-1-amine, (2R, 3 S)-3-chloro-N,N-dimethyl-2-(naphthalen-2-yl)-3-phenylpropan-1-amine, and (2S, 3R)-3-chloro-N,N-dimethyl-2-(naphthalen-2-yl)-3-phenylpropan-1-amine; or (iii) (2R, 3R)-3-chloro-N,N-dimethyl-2-(naphthalen-2-yl)-3-phenylpropan-1-amine, (2S, 3 S)-3-chloro-N,N-dimethyl-2-(naphthalen-2-yl)-3-phenylpropan-1-amine, (2R, 3 S)-3-chloro-N,N-dimethyl-2-(naphthalen-2-yl)-3-phenylpropan-1-amine, and (2S, 3R)-3-chloro-N,N-dimethyl-2-(naphthalen-2-yl)-3-phenylpropan-1-amine. The composition can comprise a pharmaceutically acceptable carrier.

In another embodiment, this document features a composition comprising, or consisting essentially of, 3-fluoro-N,N-dimethyl-2-(naphthalen-2-yl)-3-phenylpropan-1-amine or a salt thereof The 3-fluoro-N,N-dimethyl-2-(naphthalen-2-yl)-3-phenylpropan-1-amine can comprise, or can consist of, (2R, 3R)-3-fluoro-N,N-dimethyl-2-(naphthalen-2-yl)-3-phenylpropan-1-amine or (2S, 3S)-3-fluoro-N,N-dimethyl-2-(naphthalen-2-yl)-3-phenylpropan-1-amine The 3-fluoro-N,N-dimethyl-2-(naphthalen-2-yl)-3-phenylpropan-1-amine can comprise, or can consist of, (2R, 3S)-3-fluoro-N,N-dimethyl-2-(naphthalen-2-yl)-3-phenylpropan-1-amine or (2S, 3R)-3-fluoro-N,N-dimethyl-2-(naphthalen-2-yl)-3-phenylpropan-1-amine The 3-fluoro-N,N-dimethyl-2-(naphthalen-2-yl)-3-phenylpropan-1-amine can comprise, or can consist of: (i) two compounds selected from the group consisting of (2R, 3R)-3-fluoro-N,N-dimethyl-2-(naphthalen-2-yl)-3-phenylpropan-1-amine, (2S, 3S)-3-fluoro-N,N-dimethyl-2-(naphthalen-2-yl)-3-phenylpropan-1-amine, (2R, 3S)-3-fluoro-N,N-dimethyl-2-(naphthalen-2-yl)-3-phenylpropan-1-amine, and (2S, 3R)-3-fluoro-N,N-dimethyl-2-(naphthalen-2-yl)-3-phenylpropan-1-amine; or (ii) three compounds selected from the group consisting of (2R, 3R)-3-fluoro-N,N-dimethyl-2-(naphthalen-2-yl)-3-phenylpropan-1-amine, (2S, 3S)-3-fluoro-N,N-dimethyl-2-(naphthalen-2-yl)-3-phenylpropan-1-amine, (2R, 3S)-3-fluoro-N,N-dimethyl-2-(naphthalen-2-yl)-3-phenylpropan-1-amine, and (2S, 3R)-3-fluoro-N,N-dimethyl-2-(naphthalen-2-yl)-3-phenylpropan-1-amine; or (iii) (2R, 3R)-3-fluoro-N,N-dimethyl-2-(naphthalen-2-yl)-3-phenylpropan-1-amine, (2S, 3S)-3-fluoro-N,N-dimethyl-2-(naphthalen-2-yl)-3-phenylpropan-1-amine, (2R, 3S)-3-fluoro-N,N-dimethyl-2-(naphthalen-2-yl)-3-phenylpropan-1-amine, and (2S, 3R)-3-fluoro-N,N-dimethyl-2-(naphthalen-2-yl)-3-phenylpropan-1-amine The composition can comprise a pharmaceutically acceptable carrier.

In another embodiment, this document features a method for inhibiting neurotransmitter reuptake in a mammal, the method comprises, or consists essentially of, administering a composition comprising, or consisting essentially of, (a) one or more of the compounds of Table 1 or a salt thereof, (b) one or more stereoisomers of a compound of Table 1 or a salt thereof, or (c) a mixture of stereoisomers of a compound of Table 1 or salts thereof The neurotransmitter reuptake can be norepinephrine or epinephrine reuptake. The neurotransmitter reuptake can be dopamine reuptake. The neurotransmitter reuptake can be serotonin reuptake. The mammal can be a human.

In another embodiment, this document features a method for treating pain, depression, or anxiety. The method comprises, or consists essentially of, administering, to a mammal, (a) one or more of the compounds of Table 1 or a salt thereof, (b) one or more stereoisomers of a compound of Table 1 or a salt thereof, or (c) a mixture of stereoisomers of a compound of Table 1 or salts thereof.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIG. 1 is a diagram of 1-(4-fluorophenyl)-3-(methylamino)-2-(naphthalen-2-yl)propan-1-ol and its four stereoisomers.

FIG. 2 is a diagram of 1-(4-bromophenyl)-3-(methylamino)-2-phenylpropan-1-ol and its four stereoisomers.

FIG. 3 is a diagram of 3,3′-(heptane-1,7-diylbis(oxy))bis(N-methyl-2-(naphthalen-2-yl)-3-phenylpropan-1-amine) and its four stereoisomers.

FIG. 4 is a diagram of 1-(4-fluorophenyl)-3-(methylamino)-2-phenylpropan-1-ol and its four stereoisomers.

FIG. 5 is a diagram of 3-(methylamino)-1-(naphthalen-2-yl)-2-phenylpropan-1-ol and its four stereoisomers.

FIG. 6 is a diagram of 3-chloro-N,N-dimethyl-2-(naphthalen-2-yl)-3-phenylpropan-1-amine and its four stereoisomers.

FIG. 7 is a diagram of 3-fluoro-N,N-dimethyl-2-(naphthalen-2-yl)-3-phenylpropan-1-amine and its four stereoisomers.

FIG. 8 is a diagram of compounds 1a and 1b.

FIG. 9 is a diagram of Scheme 1.

FIG. 10 is a diagram of compounds 4 and 5.

FIG. 11 is a diagram of Scheme 2a.

FIG. 12 is a diagram of Scheme 2b.

FIG. 13 is a diagram of Scheme 2c.

FIG. 14 is a diagram of compounds 6, 7, 8, and 9.

FIG. 15 is a diagram of Scheme 3a.

FIG. 16 is a diagram of Scheme 3b.

FIG. 17 is a diagram of Scheme 3c.

FIG. 18 is a diagram of compounds 11, 12, 13, 14, and 15.

FIG. 19 is a diagram of Scheme 4a.

FIG. 20 is a diagram of Scheme 4b.

FIG. 21 is a diagram of Scheme 4c.

FIG. 22 is a diagram of Scheme 4d.

FIG. 23 is a diagram of Scheme 4e.

FIG. 24 is a diagram of compound 16.

FIG. 25 is a diagram of Scheme 5a.

FIG. 26 is a diagram of Scheme 5b.

### DETAILED DESCRIPTION

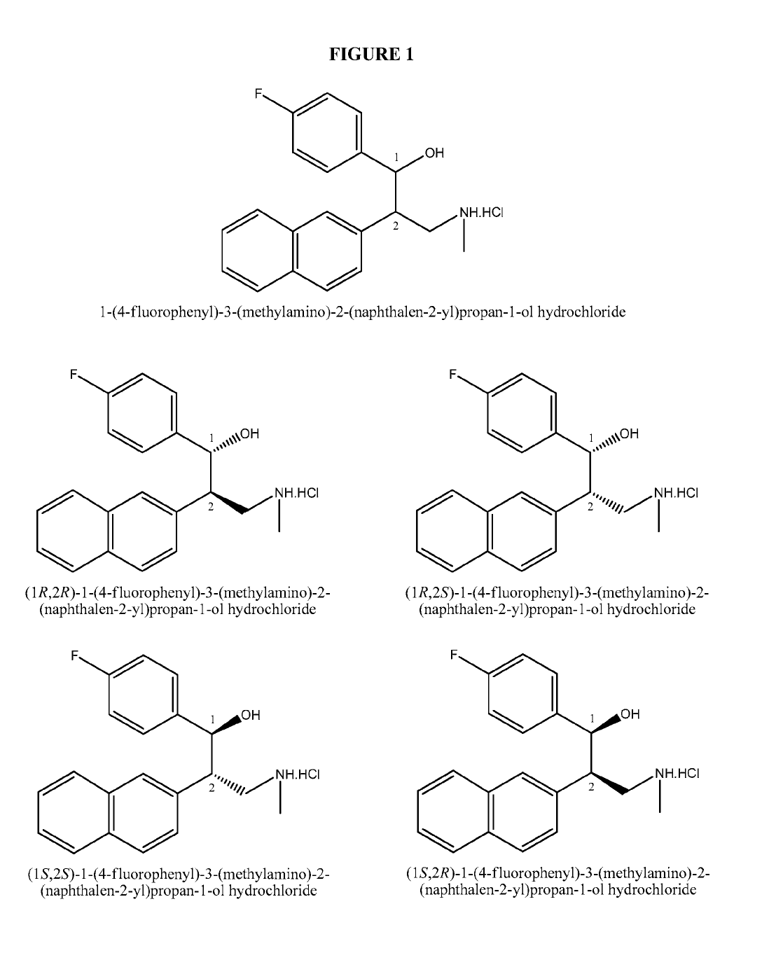

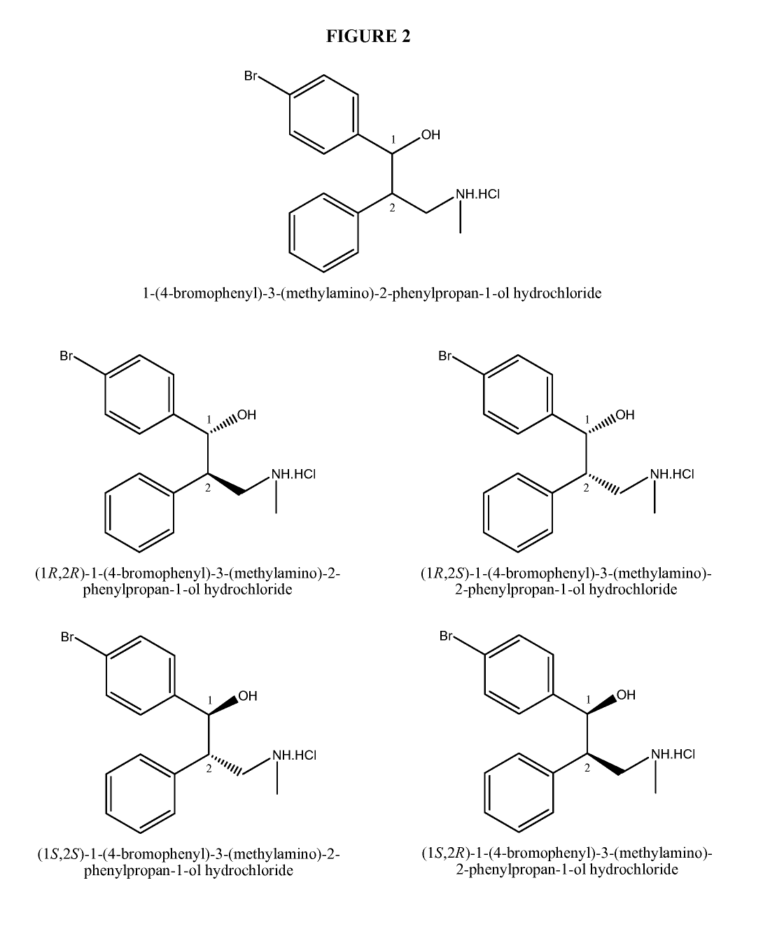

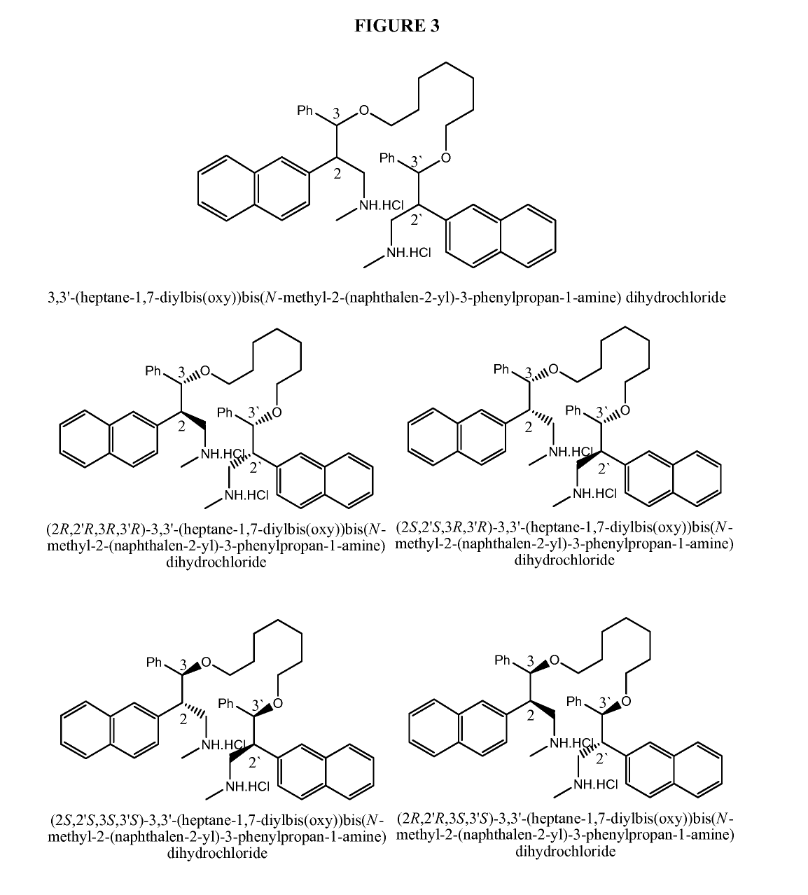

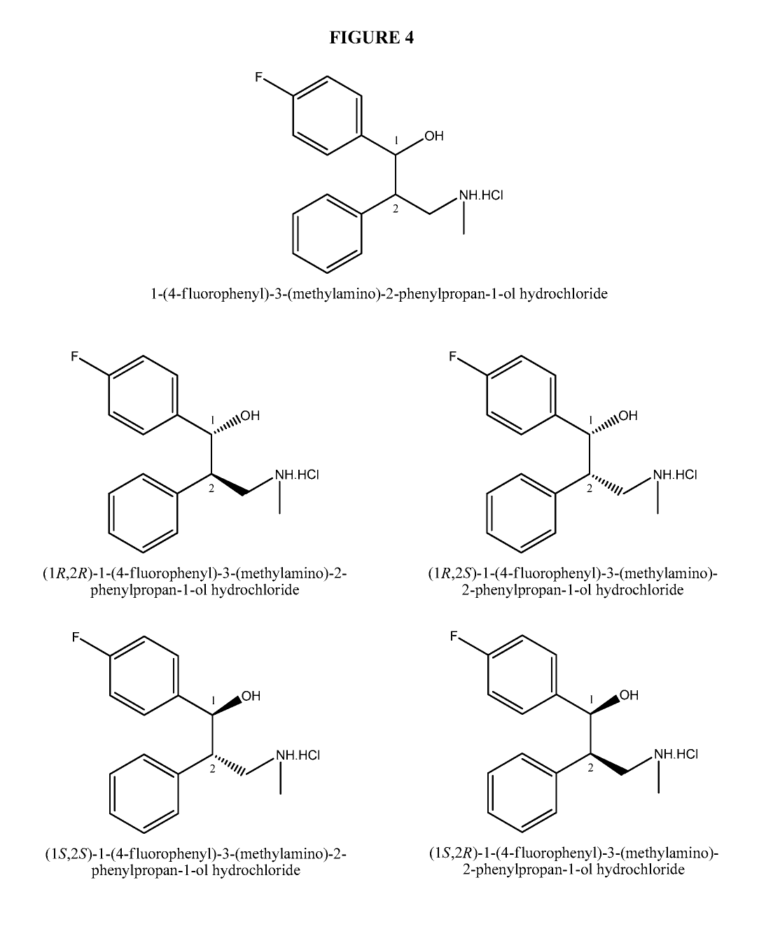

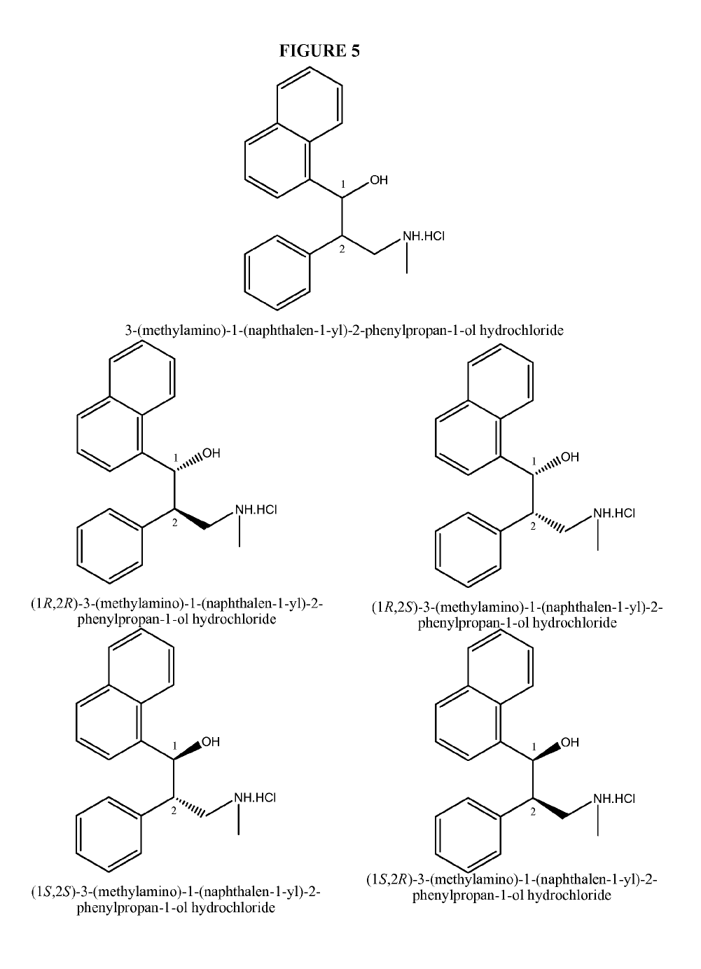

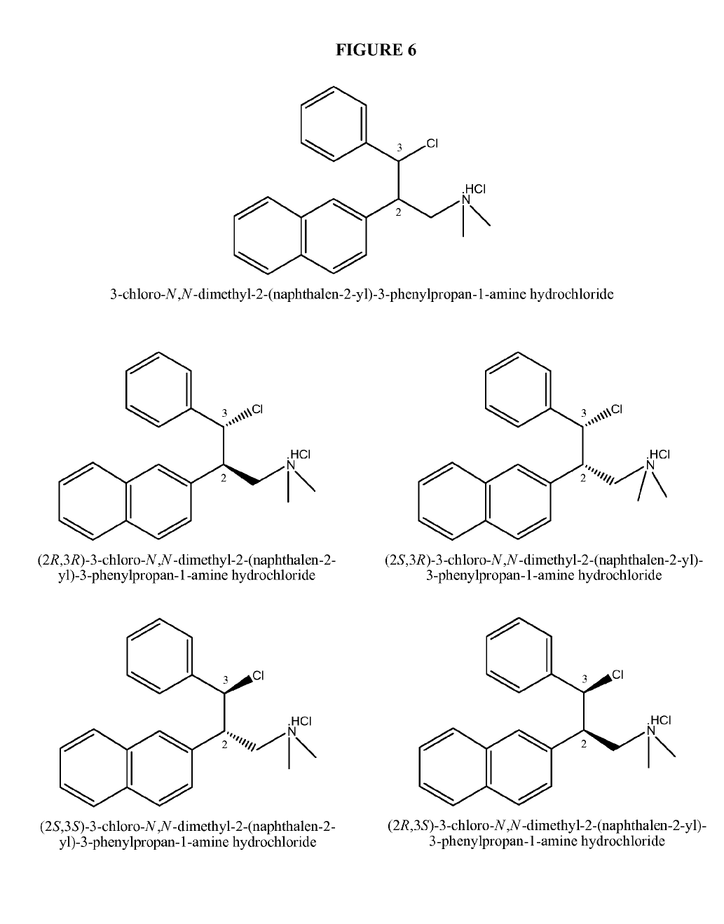

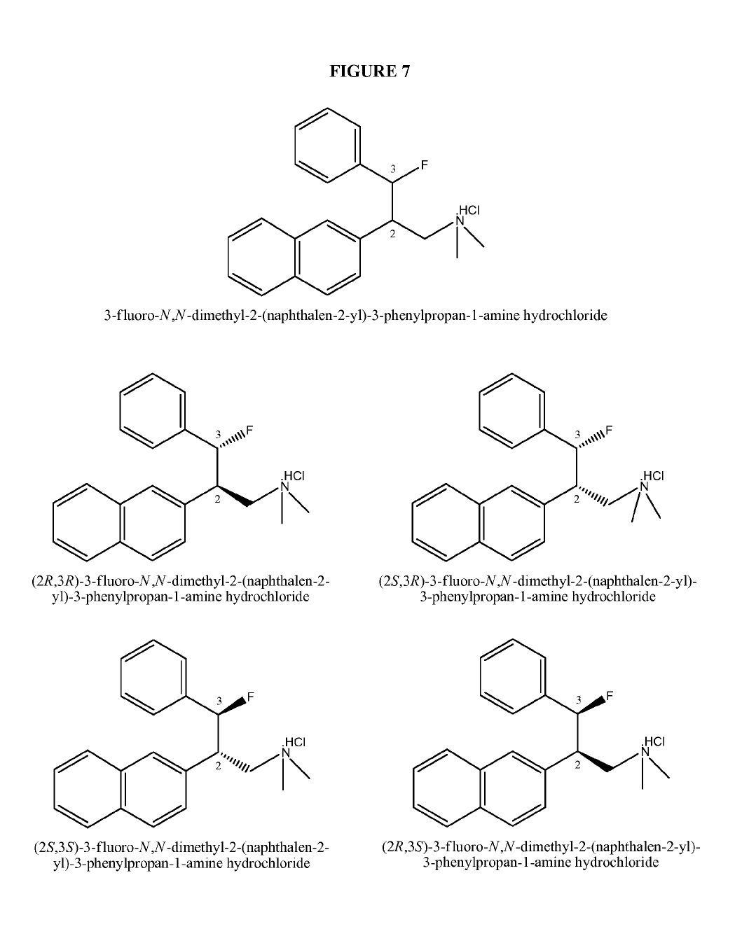

This document relates to compounds as well as methods and materials involved in modulating neurotransmitter reuptake. For example, this document provides compounds (e g, amine compounds), methods for synthesizing compounds, and methods for inhibiting neurotransmitter reuptake. Examples of compounds provided herein include, without limitation, 1-(4-fluorophenyl)-3-(methylamino)-2-(naphthalen-2-yl)propan-1-ol (FIG. 1); 1-(4-bromophenyl)-3-(methylamino)-2-phenylpropan-1-ol (FIG. 2); 3,3′-(heptane-1,7-diylbis(oxy))bis(N-methyl-2-(naphthalen-2-yl)-3-phenylpropan-1-amine) (FIG. 3); 1-(4-fluorophenyl)-3-(methylamino)-2-phenylpropan-1-ol (FIG. 4); 3-(methylamino)-1-(naphthalen-2-yl)-2-phenylpropan-1-ol (FIG. 5); 3-chloro-N,N-dimethyl-2-(naphthalen-2-yl)-3-phenylpropan-1-amine (FIGS. 6); and 3-fluoro-N,N-dimethyl-2-(naphthalen-2-yl)-3-phenylpropan-1-amine (FIG. 7).

It is understood that a particular compound can include any one of that compound's stereoisomers as well as any combination thereof. For example, a 1-(4-fluorophenyl)-3-(methylamino)-2-(naphthalen-2-yl)propan-1-ol compound can be (2R, 3R)-1-(4-fluorophenyl)-3-(methylamino)-2-(naphthalen-2-yl)propan-1-ol, (2S, 3S)-1-(4-fluorophenyl)-3-(methylamino)-2-(naphthalen-2-yl)propan-1-ol, (2R, 3S)-1-(4-fluorophenyl)-3-(methylamino)-2-(naphthalen-2-yl)propan-1-ol, or (2S, 3R)-1-(4-fluorophenyl)-3-(methylamino)-2-(naphthalen-2-yl)propan-1-ol, or any combination of (2R, 3R)-1-(4-fluorophenyl)-3-(methylamino)-2-(naphthalen-2-yl)propan-1-ol, (2S, 3S)-1-(4-fluorophenyl)-3-(methylamino)-2-(naphthalen-2-yl)propan-1-ol, (2R, 3S)-1-(4-fluorophenyl)-3-(methylamino)-2-(naphthalen-2-yl)propan-1-ol, or (2S, 3R)-1-(4-fluorophenyl)-3-(methylamino)-2-(naphthalen-2-yl)propan-1-ol. Examples of particular stereoisomers are provided in FIGS. 1-7.

This document also provides methods of synthesizing compounds such as amine compounds. For example, the compounds provided herein can be synthesized by a variety of appropriate organic chemistry techniques including, without limitation, addition of α-cyanomethylaryl anions to aryl or alkyl aldehydes to give predominantly anti-oriented β-hydroxynitriles, reduction of the resulting nitrile to a primary amine by lithium aluminum hydride or borane, conversion of the primary amine to a mono- or di-methylamine through an appropriate carbamate with reducing agents, such as lithium aluminum hydride or borane, and finally resolving the racemic amines by chiral acid-mediated optical resolution. After protection of the secondary amino function, the stereochemistry of the hydroxyl group bearing carbon can be inverted with appropriate substitution of an azide function, or any other appropriate functional group, such as a fluoride or chloride. In some cases, the hydroxyl group can be converted to a good leaving group, such as a methanesulfonate and then displaced with a fluoride, ammonia, or an ammonia derivative. A fluoride can also be directly introduced using diethylaminosulfur trifluoride (DAST). The C-3 substituted alcohol or amine can also be cylclized to a 6-membered 1,3-oxazines or 1,3-diazines through a methylene bridge after condensing with a formaldeyhyde equivalent. Other compounds with cis-stereochemistry can be isolated either as minor products from aldol-type reactions, or through Mitsunobu reactions of their major anti counterparts containing a hydroxyl group at C-3 as described herein.

### Amine Group Modification

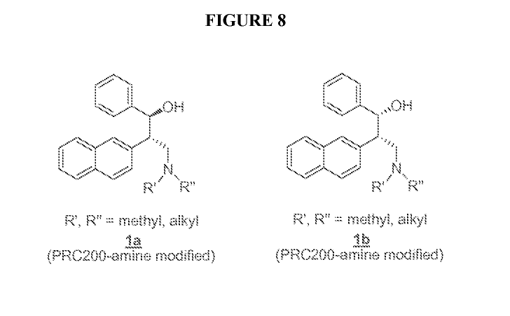

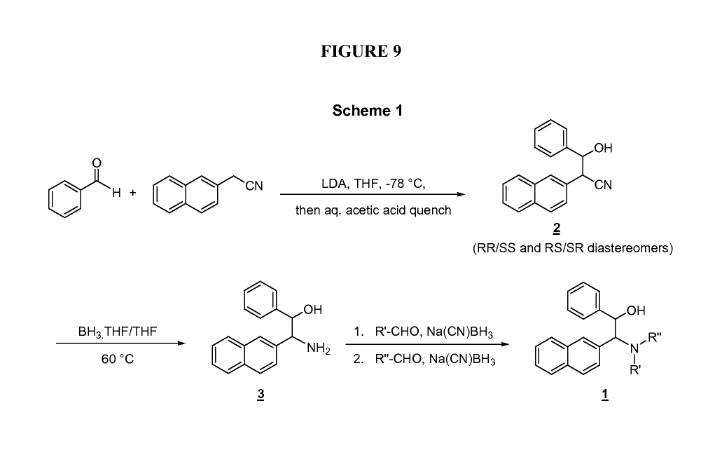

The modified 2°- and 3°-amine 1a and 1b (FIG. 8) can be prepared as set forth in Scheme 1 of FIG. 9. The reaction between the anion derived by deprotonation of β-naphthylacetonitrile with lithium diisopropylamide at −78° C. and benzaldehyde affords a mixture of two possible diastereoisomeric hydroxynitriles 2 that can be separated by selective crystallization or silica gel chromatography with the SS/RR diastereoisomer predominating. Each diastereomer can then be reduced by diborane or lithium aluminum hydride. The primary amine 3 thus obtained can be readily converted to secondary or tertiary amines as desired by reductive amination with aldehydes. The final products (secondary or tertiary amines) of this sequence of reactions can be used for making some other variants of PRC200.

### Phenyl Group Modification

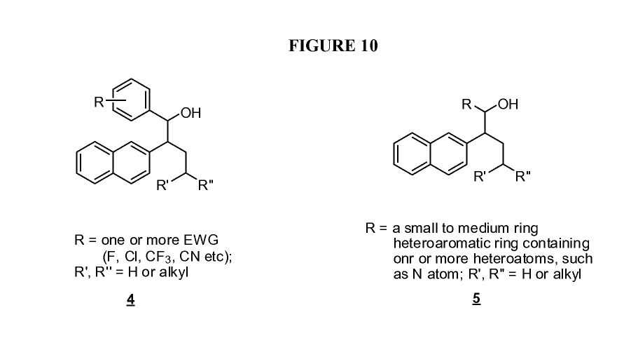

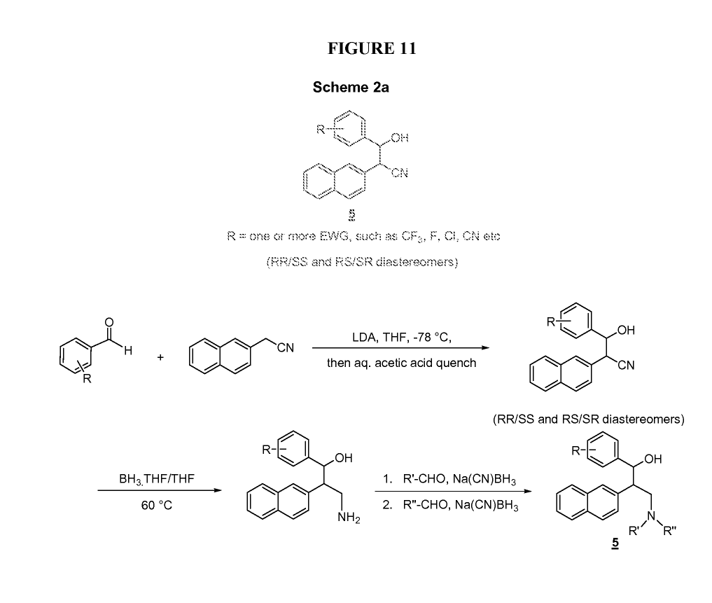

If unsubstituted phenyl ring is found to be oxidized by cytochrome-450 (CYP) isozymes to generate toxic intermediates in in vitro or in vivo assays, substitutions with electron withdrawing groups (EWG) on the phenyl rings can be made at the oxidation-prone positions. The basis synthetic strategy indicated in Scheme 2a of FIG. 11 for these PRC200 analogues 4 (FIG. 10) is the same as given in Scheme 1.

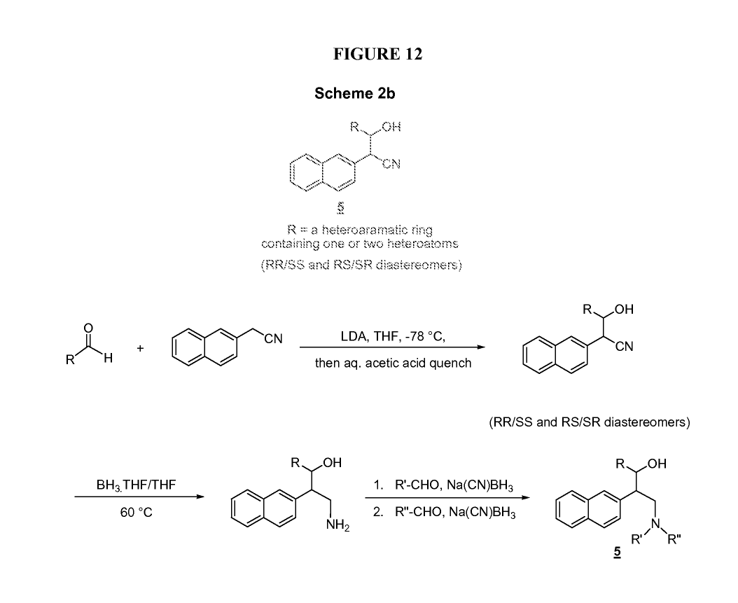

An alternative synthetic strategy can be performed where the phenyl ring can also be substituted with a heteroaromatic ring containing one or more than one heteratoms such as N, O, and S, as in the compound class 5 (FIG. 10) in Scheme 2b of FIG. 12.

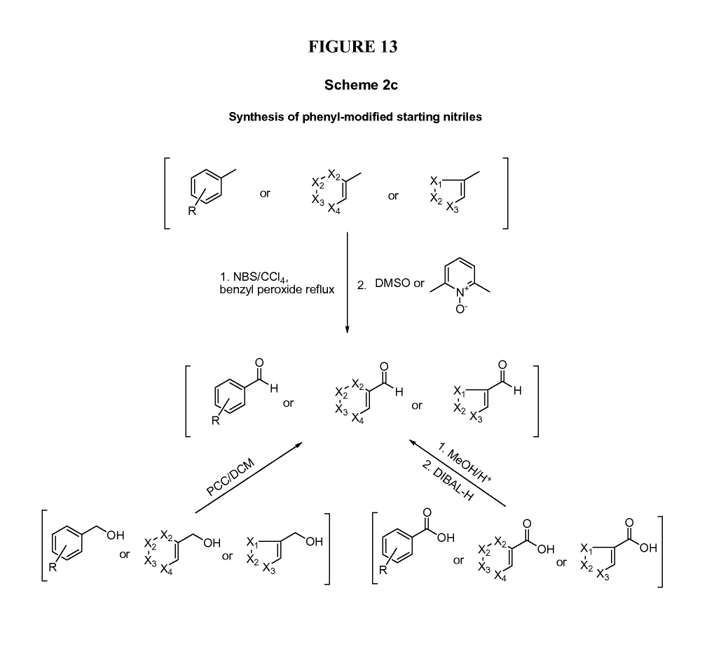

The starting aldehydes can be prepared by a number of standard synthetic schemes. For example, commercially available methyl substituted benzene or small ring heteroaromatics can be obtained and brominated by free-radical catalyzed N-bromosuccinimide (NBS) mediated reaction, and the resulting bromomethyl derivatives can be converted to aldehydes by Kornblum oxidation. Benzylic alcohols can also be directly oxidized using pyridinium chlorochromate (PCC). The required alcohols can be generated by reduction of corresponding carboxylic esters with diisobutylaluminum hydride (DIBAL-H). These synthetic conversions are given in Scheme 2c of FIG. 13.

### Naphthalene Ring Modification

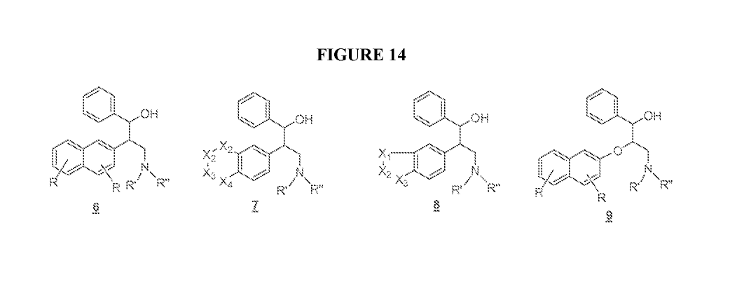

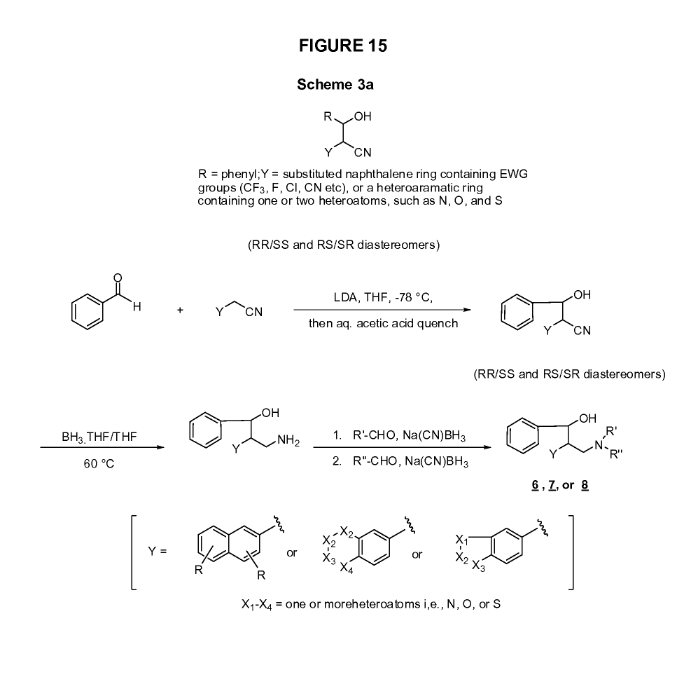

As depicted in Scheme 3a of FIG. 15, a naphthalene ring in this PRC200 analogue series can be modified in several different ways. Thus, if it is found that a specific position of the naphthalene ring is a major site of CYP isozyme oxidation generating toxic metabolites, an EWG group, such as a F, Cl, or CF₃ group can be introduced at that position to block CYP mediated oxidation as in compounds 6 (FIG. 14).

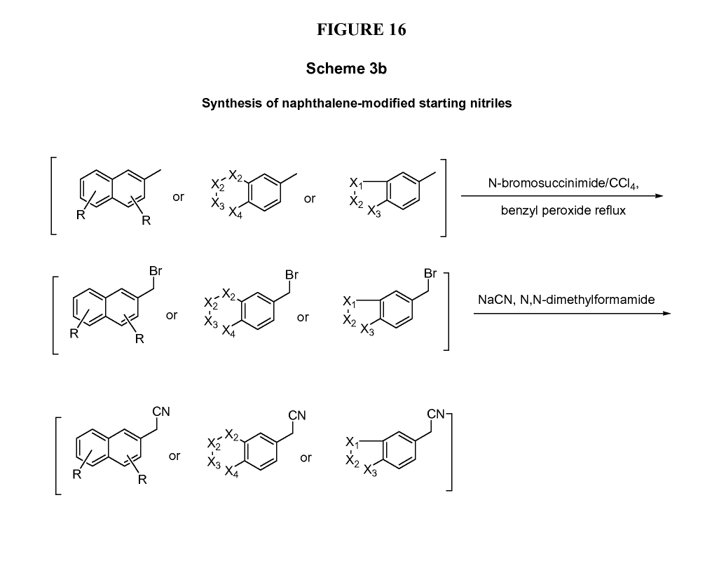

The nitrile starting materials can be prepared for synthesis of this naphthalene compound class using synthetic manipulations. One strategy can include using a methyl group on an aromatic or heteroaromatic ring of a commercially available compound and carrying out N-bromosuccinamide bromination catalyzed by a peroxide, followed by a cyanide displacement of bromine as depicted in Scheme 3b of FIG. 16.

The bulk of the naphthalene may be important for activity. If the naphthalene is the primary oxidation locus for CYP mediated oxidation, this can be replaced with a bicyclic system, such as tetralin or a phenyl-fused heterocycle, as in 6 to 8 (FIG. 14) (Scheme 3a), which may have steric bulk similar to naphthalene ring. Other bicyclic rings similar to tetralin with one or more heteroatoms in the non-benzene part can also be targeted for synthesis (Scheme 3b).

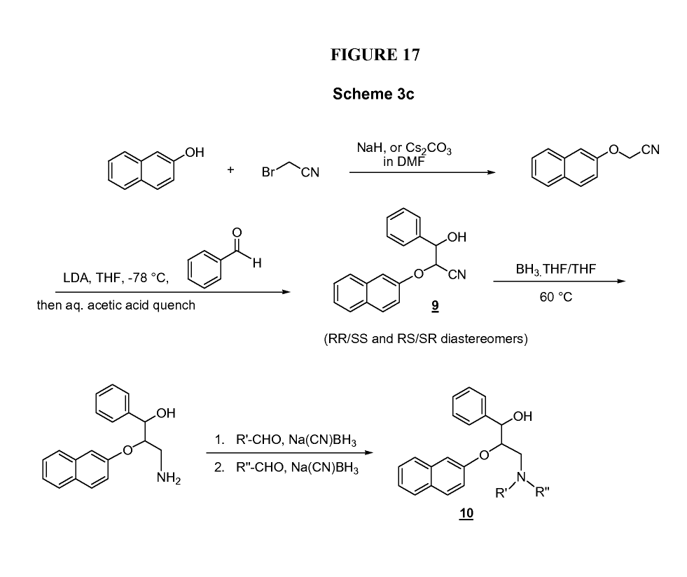

Aβ-naphthol ring, such as propranolol, can be uses as a substitution for naphthalene. A β-naphthol-containing PRC200 analogue can be synthesized as graphically indicated in Scheme 3c of FIG. 17. β-naphthol can be alkylated with a haloacetonitrile in a base catalyzed reaction to give a β-naphthyl nitrile ether which can then undergo an aldol-type reaction as in 9 intermediate that can be carried forward to 10.

### Hydroxyl Group Modification

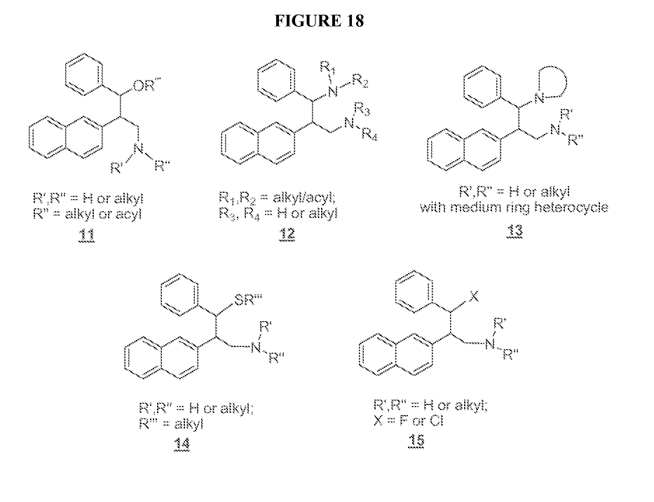

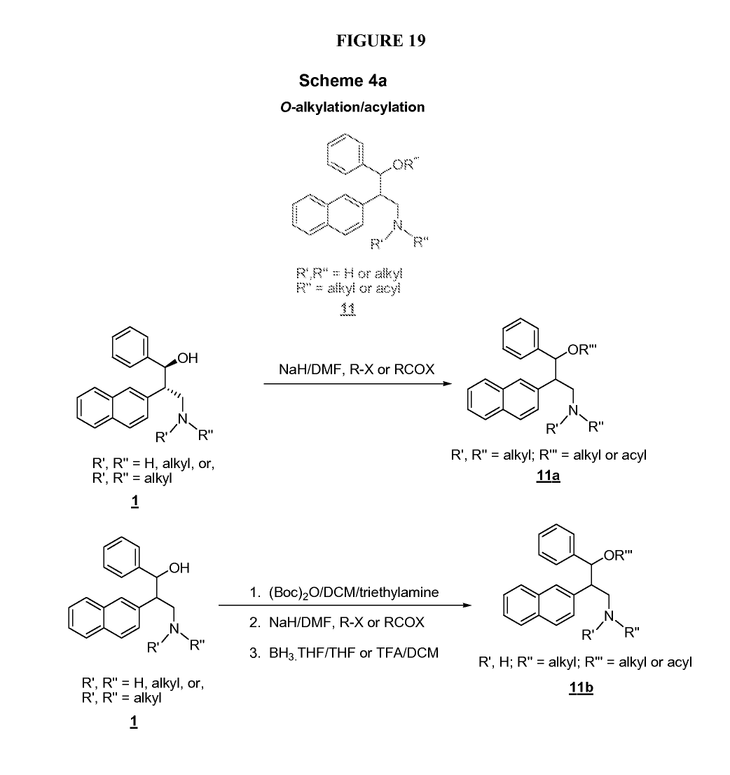

Several hydroxyl group modifications can be made. For example, medium to long O-alkylated or O-acylated analogues 11 (FIG. 18) can be synthesized using synthetic methods. For tertiary amines, the hydroxyl group can be converted to an ether, or ester, or a carbonate (Scheme 4a of FIG. 19). This OH-modification can make the analogue less prone to secondary metabolism in addition to making it more lipophilic.

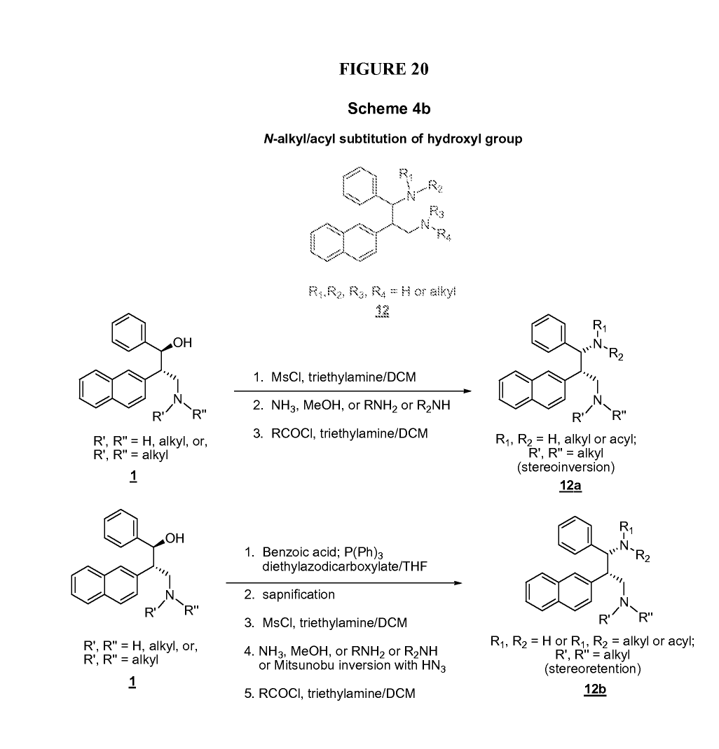

A hydroxyl group can also be replaced with either a primary amino group or any desired N-alkylated or N-acylated group with or without inversion of stereochemistry at the C-atom attached to the phenyl ring. To this end, in the tertiary amine containing PRC200 derivatives, the hydroxyl group can first be converted to a suitable good leaving group, such as a mesylate or tosylate, then the leaving group can be displaced by an S2 mechanism with a variety of secondary or tertiary amines These derivatives can also be acylated to amides 12 (Scheme 4b of FIG. 20). For retention of stereochemistry, the SS/RR diastereomer can be treated in a Mitsunobu protocol with a benzoate to give a stereochemically inverted benzoate ester which, after saponification, can be converted a mesylate, and the mesylate can carried to an amine derivative. This scenario is also represented in Scheme 4b.

Unlike the tertiary amine derivatives of PRC200 described herein, the lower secondary amines of the PRC200 may need to be protected before the chemical protocols described herein can be applied to generate the amino analogues of PRC200. This can be accomplished with a carbamate such as t-butoxycarbonyl, which can later be removed with trifluoroacetic acid.

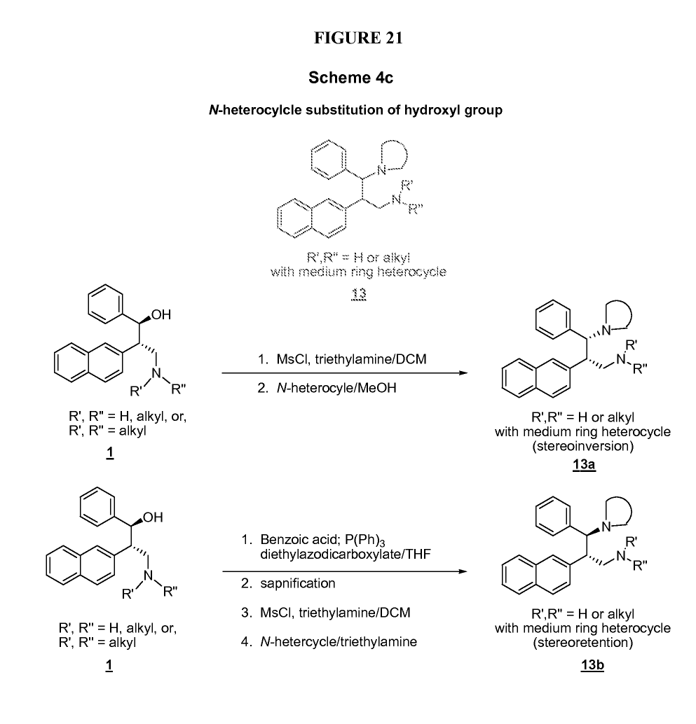

In some cases, the hydroxyl group can be replaced with or without inversion of stereochemistry with a cyclic secondary amine, such as piperidine, pyrrolidine, or morpholine, as in 13a,b (Scheme 4c of FIG. 21).

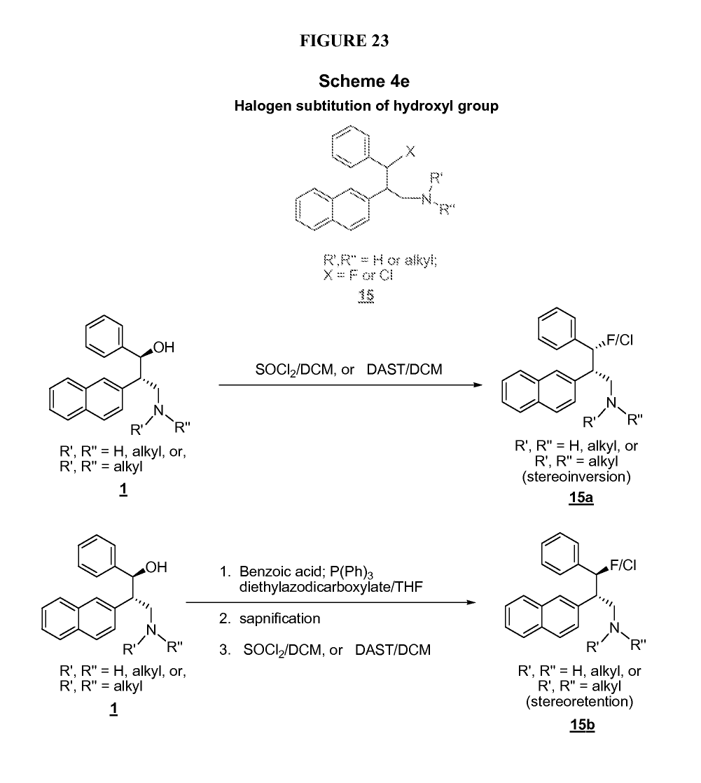

The aldol adducts can generate SS/RR diastereomer as the major isomer that can be separated from the minor isomer by either crystallization or flash silica gel chromatography. The SS/RR isomer can then be carried to the final secondary or tertiary amine stage to give compound 1 that serves as a key intermediate for making a number of analogues by hydroxyl or aromatic substitutions with electron-withdrawing (EWG), such as F, Cl, CF₃, or electron-donating (ED) groups, such as OMe and amino groups. In some cases, the stereochemistry of the hydroxyl group can be conveniently inverted by Mitsunobu protocol by reaction of the alcohol in the presence of tripheylphosphine and diethyl or diisopropyl azodicarboxylate in THF to furnish RS/SR diastereomers that can be used to make F or Cl-substituted PRC200 analogues with SS/RR or RS/SR stereochemistry (Scheme 4e of FIG. 23). These synthetic protocols can be applicable to compound series represented in Schemes 4b-d (FIGS. 20-22).

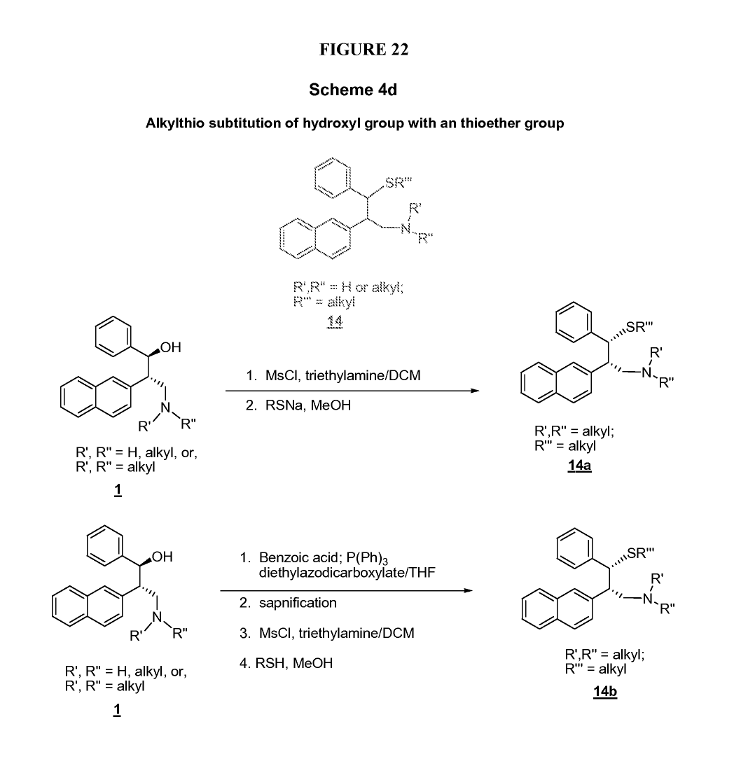

An isosteric replacement of the hydroxyl with a sulfur nucleophile can be performed as set forth in Scheme 4d of FIG. 22 where the mesylate is converted to a sulfide with an alkanethiolate.

A hydroxyl group in PRC200 or its N,N-dialkyl analogue can be substituted with a fluoro group or a chloro group. A fluoro group is an isostere of a hydroxyl group because of its similar electronegativity and size constraints. These groups can be installed by reaction of a hydroxyl group with either thionyl chloride (for a chloro substituent) or with N,N-diethylsulfurtrifluoride (for a fluoro substituent) as set forth in Scheme 4e of FIG. 23.

A F- and Cl-substitution of a hydroxyl can also be carried out on the PRC200 ring variants with EWG's and ED's.

Modification of Phenyl and/or Naphthalene Ring with Electron-Donating (ED) Groups

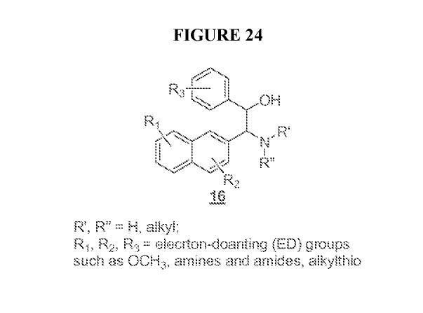

ED groups can be introduced on the phenyl and naphthyl rings to improve potency or selectivity of the analogues as in the compound series 16 (FIG. 24). The ED groups can be added at the oxidation-prone positions of the aromatic rings of PRC200 analogues if those positions become known by tandem mass spectrometry analysis.

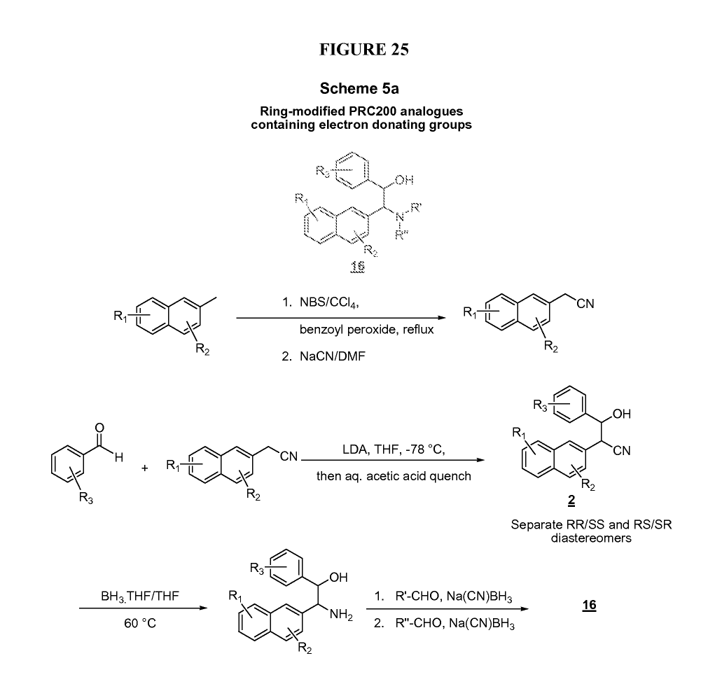

The starting aldehydes and naphthalene acetonitrile derivatives for completion of the synthesis of 16 can be fashioned from either commercially available materials carrying either a methyl group or an alcohol or acid by standard synthetic manipulations. A method for preparing these derivatives is set forth in Scheme 5a of FIG. 25.

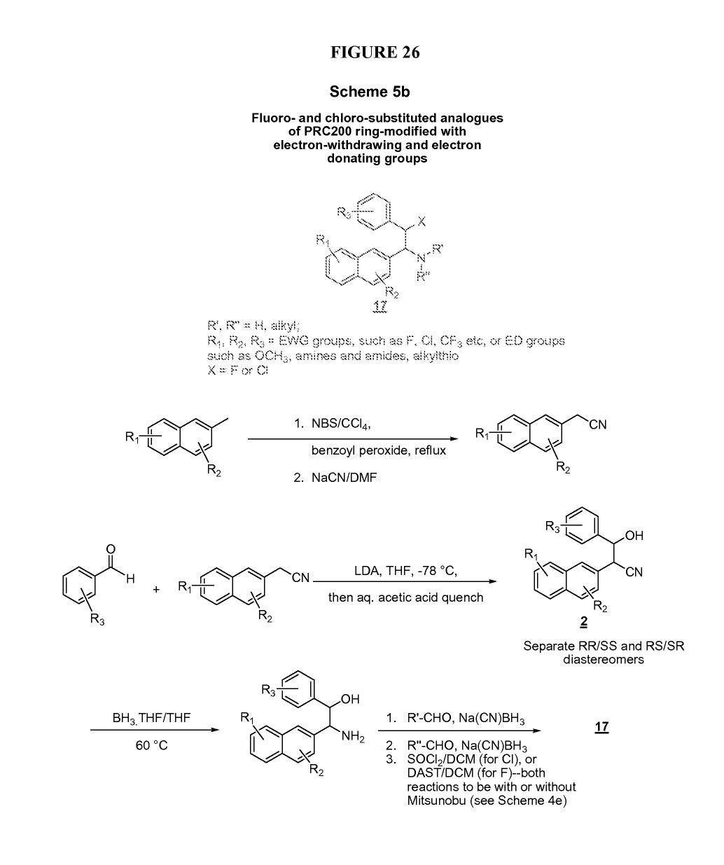

Other hydroxyl group-modified analogues, similar to those described in Scheme 4e with halogen substitution, can be synthesized with EWG- or ED-permutations around the phenyl and/or naphthalene rings. This compound series 17 is set forth in Scheme 5b of FIG. 26.

### Compounds

Any compound provided herein, which can be active in triple reuptake inhibition assays, can be resolved by diastereomeric bias on acid-base salt formation with optically pure acids, such as tartaric acid, lactic acid, and camphorsulfonic acid.

Any compound provided herein can be a mixture of stereoisomers or can be resolved to form a racemic syn-diastereomer composition, or a racemic anti-diastereomer composition, or these racemates can be optically resolved to furnish pure enantiomers. For example, a compound can be resolved to a pure enantiomer by classical resolution using enantiomerically pure acids including, without limitation, (+)- and (−)-tartaric acid, (+)- and (−)-ditoluyl-tartaric acid, (+)- and (−)-camphorsulfonic acid, or any other optically pure acids.

Any appropriate method can be used to isolate diastereomers and enantiomers such as those described elsewhere (Eliel et al., In: Stereochemistry of Organic Compounds; John Wiley & Sons: New York, 1994). The racemic anti-diastereomeric mixture (50:50 of the 2S,3S and 2R,3R enantiomers) of a compound or derivative provided herein can be resolved into the pure enantiomers by classical optical resolution methods. For example, a racemic anti-diastereomer of 3-chloro-N,N-dimethyl-2-(naphthalen-2-yl)-3-phenylpropan-1-amine (50:50 2R,3R and 2S,3S;

FIG. 6) can be isolated by selective crystallization from a diastereomeric salt mixture with a chiral organic acid, such as tartaric acid, camforsulfonic acid or a lactic acid etc. After selective crystallization, the optically enriched or pure enantionmeric forms can be obtained by treatment with an aqueous strong base.

Any compound or enantiomer thereof provided herein can be chemically converted from its free base form to a pharmaceutically acceptable salt by reacting the free base with an equivalent amount of any acid that forms a non-toxic salt. Such acids can be either inorganic or organic including, without limitation, hydrochloric acid, hydrobromic acid, fumaric acid, maleic acid, succinic acid, sulfuric acid, phosphoric acid, tartaric acid, acetic acid, citric acid, and oxalic acid. Any compound or pharmaceutically acceptable salt thereof provided herein can be administered to a mammal by itself or in combination with a carrier. Such carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Preservatives, flavorings, and other additives such as, for example, antimicrobials, anti-oxidants, chelating agents, inert gases, and the like can also be present. It will be appreciated that any compound provided herein that is to be administered to a mammal can contain zero, one, or more than one commonly known pharmaceutically acceptable carriers.

### Inhibiting Neurotransmitter Reuptake

This document provides methods for using the compounds provided herein to inhibit neurotransmitter reuptake in a mammal The term “inhibit” as used herein with respect to neurotransmitter reuptake refers to any reduction in neurotransmitter reuptake. For example, a reduction in neurotransmitter reuptake greater than zero percent (e.g., greater than 0.1, 0.5, 1, 2, 5, 10, 25, 50, 75, or 99 percent) is considered an inhibition of neurotransmitter reuptake. In some embodiments, a compound provided herein can inhibit neurotransmitter reuptake such that the reduction in neurotransmitter reuptake is greater than zero percent (e.g., greater than 0.1, 0.5, 1, 2, 5, 10, 25, 50, 65, 75, 85, 95, or 99 percent) as compared to untreated controls (e.g., untreated mammals or cells). Any appropriate method can be used to assess whether or not neurotransmitter reuptake has been inhibited in a mammal Such methods can be qualitative or quantitative. An example of a qualitative method includes assessing whether or not a mammal with depression experiences loss of pleasure in daily activities, significant weight loss or gain, changes in mobility (e.g., lethargy, nervousness), feelings of worthlessness, diminished ability to concentrate, or suicidal thoughts to a lesser extent following treatment with a compound provided herein than the extent experienced before treatment. In some cases, such methods can be quantitative. For example, the concentration of serotonin in a platelet sample from a mammal after treatment with a compound can be measured and compared to the concentration of serotonin in a platelet sample from the same mammal before treatment with that compound. If the concentration of serotonin after treatment is reduced compared to the concentration of serotonin before treatment, then that compound inhibited neurotransmitter reuptake in that mammal

To inhibit neurotransmitter reuptake, an effective amount of any compound provided herein can be administered to a mammal The term “effective” as used herein refers to any amount that induces a desired level of neurotransmitter reuptake inhibition while not inducing significant toxicity in the mammal Such an amount can be determined using the methods and materials provided herein. An effective amount of a compound or formulation containing a compound can be any amount that reduces, prevents, or eliminates an anxiety or depressive disorder or relieves pain upon administration to a mammal without producing significant toxicity to that mammal Some compounds may have a relatively broad concentration range that is effective while others may have a relatively narrow effective concentration range. In addition, the effective amount can vary depending upon the specific mammal or the specific anxiety or depressive disorder to be treated because certain mammals and anxiety or depressive disorders can be more or less responsive to a particular compound. Such effective amounts can be determined for individual compounds using commonly available or easily ascertainable information involving equilibrium dissociation constants, mammal toxicity concentrations, and bioavailability. For example, non-toxic compounds typically can be directly or indirectly administered to a mammal in any amount that reduces, prevents, or eliminates an anxiety or depressive disorder in that mammal Using the information provided herein, such effective amounts can also be determined by routine experimentation in vitro or in vivo. For example, a patient having an anxiety or depressive disorder can receive direct administration of a compound provided herein in an amount to achieve a blood level close to the equilibrium dissociation constant (i.e., K_{d}) calculated from in vitro analysis sufficient to inhibit the uptake of a particular neurotransmitter. If the patient fails to respond, then the amount can be increased by, for example, two fold. After receiving this higher concentration, the patient can be monitored for both responsiveness to the treatment and toxicity symptoms, as well as blood levels of the drug, and adjustments made accordingly.

To help determine effective amounts of different compounds, it can be useful to refer to an effective amount equivalent based on the effective amount of a common drug used to treat anxiety or depressive disorders. For example, the direct administration of 0.30 mg/kg Prozac® (fluoxetine) daily for three weeks to a mammal can be an effective amount for treating anxiety or depressive disorders. The effects produced by this effective amount can be used as a reference point to compare the effects observed for other compounds used at varying concentrations. Once an equivalent effect is observed, then the specific effective amount for that particular compound can be determined In this case, that particular amount would be termed a Prozac® effective amount equivalent.

The ability of a compound to inhibit neurotransmitter reuptake also can be assessed in vitro. For example, the level of serotonin reuptake can be determined by measuring the amount of radiolabeled serotonin taken up by synaptosomes (“pinched-off” nerve endings) purified from a tissue source abundant in serotonin transporters (e.g., rat brain cortical tissue). Rat brain cortical tissue can be isolated to produce neuronal membrane fragments such that the membrane fragments close back on themselves to form synaptosomes that retain functional serotonin transporters. The serotonin transporters concentrate serotonin by transporting it from the fluid in which the synaptosomes are suspended to the interior of the synaptosomes. If the serotonin in the suspension fluid is radiolabeled, then the level of serotonin reuptake can be measured by counting the radioactivity in the synaptosomal pellet obtained by rapid filtration or centrifugation. The ability of a compound to inhibit the level of serotonin reuptake can be determined by adding different concentrations to aliquots of the same synaptosomal preparation. For example, the potency of 3-chloro-N,N-dimethyl-2-(naphthalen-2-yl)-3-phenylpropan-1-amine as an inhibitor of serotonin reuptake can be measured by (1) adding different concentrations of 3-chloro-N,N-dimethyl-2-(naphthalen-2-yl)-3-phenylpropan-1-amine to aliquots of synaptosomes purified from rat brain cortical tissue, (2) adding the same concentration of radiolabeled serotonin to each aliquot, (3) allowing the serotonin transporters to concentrate the radiolabeled serotonin in the synaptosomes, and (4) counting the radioactivity in the synaptosomal pellet of each aliquot obtained after centrifugation. Compounds with a higher potency will more effectively inhibit reuptake at lower concentrations thus resulting in less detectable radioactivity in the synaptosomal pellet.

In another in vitro example, intact cultured mammalian cells expressing a particular recombinant neurotransmitter transporter can be used to assess the ability of a compound to inhibit neurotransmitter reuptake. For example, the potency of 3-chloro-N,N-dimethyl-2-(naphthalen-2-yl)-3-phenylpropan-1-amine as an inhibitor of norepinephrine transport can be measured using cultured mammalian cells expressing the norepinephrine transporter. In addition, the potency of a particular compound to inhibit multiple neurotransmitter transporters can be measured. For example, the potency of 3-chloro-N,N-dimethyl-2-(naphthalen-2-yl)-3-phenylpropan-1-amine as an inhibitor of both serotonin and norepinephrine transport can be measured using separate cultured mammalian cells expressing the serotonin transporter and cultured mammalian cells expressing the norepinephrine transporter. It is understood that measured neurotransmitter transport levels are compared to controls. Controls include, without limitation, vehicle only as well as known inhibitors such as Prozac®, Paxil® (paroxetine), Effexor® (venlafaxine), or Norpramin® (desipramine).

In addition, the potency of a compound to inhibit the reuptake of different neurotransmitters can be assessed by determining the equilibrium dissociation constant (i.e., K_{d}) of that particular compound for a particular neurotransmitter transporter. Typically, the K_{d} value is determined as described elsewhere (Tatsumi et al, Eur. J. Pharmacol., 340:249-258 (1997)). Once determined, the K_{d} value for a particular compound can be used to compare that compound's potency with the potency of other compounds or other known inhibitors. For example, if a particular compound has a K_{d} of 4.1 nM for the serotonin transporter and a K_{d} of 12.5 nM for the norepinephrine transporter, then that particular compound can be characterized as having a greater ability to inhibit serotonin reuptake compared to norepinephrine reuptake. Likewise, if a first compound has a K_{d} of 54 nM for the dopamine transporter and a second compound has a K_{d} of 134 nM for the dopamine transporter, then the first compound can be characterized as having a greater ability to inhibit dopamine reuptake compared to the second compound.

Various factors can influence the actual effective amount used for a particular application. For example, the frequency of administration, duration of treatment, rate of metabolism of the drug, combination of other compounds, and site of administration may require an increase or decrease in the actual effective amount administered.

The frequency of administration can be any frequency that reduces, prevents, or eliminates an anxiety disorder or depression in a mammal without producing significant toxicity to the mammal For example, the frequency of administration can be from about once a day to about once a month, or more specifically, from about twice a day to about once a week. In addition, the frequency of administration can remain constant or can be variable during the duration of treatment. As with the effective amount, various factors can influence the actual frequency of administration used for a particular application. For example, the effective amount, duration of treatment, rate of metabolism of the drug, combination of other compounds, and site of administration may require an increase or decrease in administration frequency.

An effective duration for amine compound administration can be any duration that reduces, prevents, or eliminates an anxiety or depressive disorder in a mammal without producing significant toxicity to the mammal Thus, the effective duration can vary from several days to several weeks, months, or years. In general, the effective duration for the treatment of an anxiety or depressive disorder can range in duration from several days to several years. Once the compound administrations are stopped, however, the treated anxiety or depressive disorder may return. Thus, the effective duration for the prevention of an anxiety or depressive disorder can last in some cases for as long as the individual is alive.

Multiple factors can influence the actual effective duration used for a particular treatment or prevention regimen. For example, an effective duration can vary with the frequency of compound administration, effective compound amount, combination of multiple compounds, and site of administration. It is noted that diagnostic algorithm methods can be devised to determine or reflect appropriate effective doses, durations, and frequencies.

The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity. Significant toxicity can vary for each particular mammal and each particular composition.

Any combination of compounds provided herein can be administered to a mammal For example, two compounds can be administered together to a mammal to inhibit norepinephrine reuptake in that mammal In another example, one or more compounds that can inhibit serotonin reuptake and one or more compounds that can inhibit dopamine reuptake can be administered together to a mammal to inhibit both serotonin and dopamine reuptake in that mammal The efficacy of such combinations can be assessed using the methods and materials provided herein.

A compound or combination of compounds provided herein can be administered to any part of a mammal's body. For example, a compound can be delivered to, without limitation, spinal fluid, blood, lungs, intestines, muscle tissues, skin, joints, peritoneal cavity, or brain of a mammal In addition, a compound or combination of compounds can be administered intravenously, intraperitoneally, intramuscularly, subcutaneously, intrathecally, intracerebroventricularly, or intradermally, orally, by inhalation, or by gradual perfusion over time. The duration of treatment can be any length of time from as short as one day to as long as the life span of the mammal (e.g., many years). For example, a compound provided herein can be administered daily for three months or ten years. It is also noted that the frequency of treatment can be variable. For example, a compound can be administered once (or twice, three times, etc.) daily, weekly, monthly, or yearly.

The invention will be further described in the following examples, which do not limit the scope of the invention described in the claims.

### EXAMPLES

### Example 1

### Human Transporter Binding Studies

Human embryonic kidney (HEK-293) cells stably transfected and constitutively expressing the human norepinephrine transporter (hNET; Pacholczyk et al., Nature, 350:350-354 (1991)), the human dopamine transporter (hDAT; Pristupa et al., Mol. Pharmacol., 45:125-135 (1994)), or the human serotonin transporter (hSERT; Ramamoorthy et al., Proc. Natl. Acad. Sci. U. S. A. 90:2542-2546 (1993)) were grown and passaged in 150-mm Petri dishes with 17.5 mL of medium. HEK-293 cells expressing the recombinant hSERT or hDAT were incubated with Dulbecco's modified Eagles medium (MEM) supplemented with 0.1 mM nonessential amino acid solution for MEM, 5% v/v fetal bovine serum and 1 U/mL penicillin/streptomycin solution. Cells expressing the hNET transporter were incubated with Dulbecco's modified Eagles medium supplemented with 10% v/v fetal bovine serum and 100 U/mL penicillin/streptomycin solution and 2 mM L-glutamine. All cells were grown until 70-80% confluent on 175 cm² flasks in a humidified 10% CO₂/90% air environment at 37° C., with the selecting antibiotic Geneticin sulfate at 250 ng/mL. The cells were incubated in 10% CO₂, 90% air at 37° C. and 100% humidity.

Cell membranes containing hSERT, hNET, or hDAT were prepared from the cell lines to assay ligand binding for each of the transporters. Briefly, the cell medium was removed by aspiration, and the cells were washed with 4 mL modified Puck's D1 solution (solution 1; Richelson et al. in “Methods in Neurotransmitter Receptor Analysis” Yamamura, H. I.; Enna, S. J.; Kuhar, M. J. Eds.; New York, Raven Press, 1990, pp 147-175). The washed cells were incubated for 5 minutes at 37° C. in 10 mL solution 1 containing 100 mM ethylene glycol-bis N,N,N′,N′-tetraacetic acid (EGTA). The cells were then scraped from the flask surface with a rubber spatula, placed into a centrifuge tube, and collected by centrifugation at 1000×g for 5 minutes at 4° C. The resulting supernatant was discarded, and the cell pellet was resuspended in 0.5 to1.0 mL of the appropriate binding buffer (described below). The resuspended cell pellet was homogenized using a Polytron for 10 seconds at setting 6. The resulting homogenate was centrifuged at about 36,000×g for 10 minutes at 4° C. The supernatant was discarded, and the pellet was resuspended in the same volume of the appropriate binding buffer and centrifuged again. The supernatant was discarded, and the final pellet containing cell membranes was resuspended in the appropriate binding buffer and stored at −80° C. until use. The final protein concentration was determined by the Lowry assay using bovine serum albumin as a standard (Lowry et al., J. Biol. Chem. 193:265-275 (1951)).

Radioligand binding assays for the indicated transporters were performed as follows. To assess binding to the cloned hSERT, cells expressing hSERT were homogenized in 50 mM Tris-HCl with 120 mM NaCl and 5 mM KCl (pH 7.4). The binding reaction consisted of 10 μg cell membrane protein, 1.0 nM [³H]citalopram (citalopram, [N-methyl-³H], specific activity 79.0 Ci/mmol; PerkinElmer, Boston, Mass.), and varying concentrations of either unlabeled citalopram or the test compound. A reaction to determine non-specific binding consisted of 10 μg cell membrane protein, 0.5 nM [³H]citalopram, and 1 μM final concentration of unlabeled citalopram. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman-Coulter, Fullerton, Calif.). Radioactivity was measured with a Beckman liquid scintillation counter (LS 6000TA).

To assess binding to the cloned hNET, cells expressing hNET were homogenized in 50 mM Tris-HCl with 300 mM NaCl and 5 mM KCl (pH 7.4). The binding reaction consisted of 5 μg cell membrane protein, 0.5 nM [³H]nisoxetine (nisoxetine HCl, [N-methyl-³H], specific activity 82.0 Ci/mmol; Amersham, Arlington Hts., Ill.), and varying concentrations of either unlabeled nisoxetine or the test compound. A reaction to determine non-specific binding consisted of 5 μg cell membrane protein, 0.5 nM [³H]nisoxetine, and 1 μM final concentration of unlabeled nisoxetine. The reactions were incubated at 22° C. for 60 minutes. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman-Coulter, Fullerton, Calif.). Radioactivity was measured with a Beckman liquid scintillation counter (LS 6000TA).

To assess binding to the cloned hDAT, cells expressing hDAT were homogenized in 50 mM Tris-HCl with 120 mM NaCl (pH 7.4). The binding reaction contained 15 μg cell membrane protein, 1 nM [³H]WIN35428 (WIN35428, [N-methyl-³H], specific activity 85.9 Ci/mmol; PerkinElmer, Boston, Mass.), and varying concentrations of either unlabeled WIN35428 or the test compound. A reaction to determine non-specific binding contained 15 μg cell membrane protein, 1 nM [³H]WIN35428, and 10 μM final concentration of unlabeled WIN35428. The reactions were incubated at 22° C. for 1 hour. Following incubation, the reactions were terminated by rapid filtration through separate GF/B filter strips pretreated with 0.2% polyethylenimine in a 48-well Brandel cell harvester. The cell membrane-containing filter strips were then rinsed five times with ice-cold 0.9% NaCl. After rinsing, individual filters were cut from the strip and placed in a scintillation vial containing 6.5 mL of Redi-Safe (Beckman-Coulter, Fullerton, Calif.). Radioactivity was measured with a Beckman liquid scintillation counter (LS 6000TA).

Following the radioligand binding assays, the data were analyzed using the

LIGAND program (Munson and Rodbard, Analyt. Biochem., 107:220-239 (1980)) to provide values for the equilibrium dissociation constants (K_{d}). The program was modified to calculate the Hill coefficient (nH). Data are presented as geometric mean±S.E.M. of at least three independent experiments. One-component models and two-component models were compared using the root mean square error of each fit and the F test. A low K_{d} for a compound indicates strong binding to the transporter (i.e., reuptake inhibition).

HCl salts of the test compounds listed in Table 1 were made and tested as described herein.

TABLE 1
Test compounds.

| | | Molecular |
|---|---|---|
| Compound Name | Simple Name | Weight |
| (1S,2S)-3-(methylamino)-2- | PRC200-SS | 327.85 |
| (naphthalen-2-yl)-1- | | |
| phenylpropan-1-ol | | |
| (1R,2S)-1-(4-fluorophenyl)- | MCJ001-4FPh—OH- | 345.84 |
| 3-(methylamino)-2- | RS/SR | |
| (naphthalen-2-yl)propan-1-ol | | |
| (1S,2S)-1-(4-fluorophenyl)- | MCJ001-4FPh—OH- | 345.84 |
| 3-(methylamino)-2- | SS/RR | |
| (naphthalen-2-yl)propan-1-ol | | |
| (1R,2S/1S,2R)-1-(4- | MCJ001-Br—Ph—OH- | 356.69 |
| bromophenyl)-3- | RS/SR | |
| (methylamino)-2- | | |
| phenylpropan-1-ol | | |
| (1S,2S/1R,2R)-1-(4- | MCJ001-Br—Ph—OH- | 356.69 |
| bromophenyl)-3- | SS/RR | |
| (methylamino)-2- | | |
| phenylpropan-1-ol | | |
| (2S,2′S,3R,3′R)-3,3′- | MCJ001-Dimer7-SS | 750.37 |
| (heptane-1,7- | | |
| diylbis(oxy))bis(N-methyl-2- | | |
| (naphthalen-2-yl)-3- | | |
| phenylpropan-1-amine) | | |
| (1R,2S)-1-(4-fluorophenyl)- | MCJ001-F—Ph—OH- | 295.78 |
| 3-(methylamino)-2- | RS/SR | |
| phenylpropan-1-ol | | |
| (1S,2S)-1-(4-fluorophenyl)- | MCJ001-F—Ph—OH- | 295.78 |
| 3-(methylamino)-2- | SS/RR | |
| phenylpropan-1-ol | | |
| (1R,2S/1S,2R)-3- | MCJ001-NA-Ph—OH- | 327.84 |
| (methylamino)-1- | RS/SR | |
| (naphthalen-2-yl)-2- | | |
| phenylpropan-1-ol | | |
| (1S,1R/2S,2R)-3- | MCJ001-NA-Ph—OH- | 327.84 |
| (methylamino)-1- | SS/RR | |
| (naphthalen-2-yl)-2- | | |
| phenylpropan-1-ol | | |
| (2S,3R)-3-chloro-N,N- | MCJ002-Cl-RS | 360.3 |
| dimethyl-2-(naphthalen-2- | | |
| yl)-3-phenylpropan-1-amine | | |
| (2S,3S)-3-chloro-N,N- | MCJ002-Cl-SS | 360.3 |
| dimethyl-2-(naphthalen-2- | | |
| yl)-3-phenylpropan-1-amine | | |
| (2S,3R)-3-fluoro-N,N- | MCJ002-F-RS | 343.87 |
| dimethyl-2-(naphthalen-2- | | |
| yl)-3-phenylpropan-1-amine | | |

Human transporter binding data are presented in Table 2 for the test compounds listed in Table 1.

TABLE 2
Human transporter binding data.

| | Equilibrium Dissociation Constant (K_{D}) for | | | | |
|---|---|---|---|---|---|
| | Transporter Binding (Geometric Mean; nM ± SEM)^{a} | | | | Potency |
| Simple Name | hSERT (S) | hNET (N) | hDAT (D) | Potency | Range |
| PRC200-SS | 3.8 ± 0.3 | 0.6 ± 0.2 | 27 ± 3 | N > S > D | 45 |
| MCJ001-4F—Ph—OH-RS/SR | 27 ± 4 | 49 ± 4 | 250 ± 40 | S > N > D | 9 |
| MCJ001-4F—Ph—OH-SS/RR | 33 ± 5 | 11 ± 1 | 90 ± 10 | N > S > D | 8 |
| MCJ001-Br—Ph—OH-RS/SR | 3320^{b} | 4500^{b} | 1520^{b} | D > S > N | 3 |
| MCJ001-Br—Ph—OH-SS/RR | 7300^{b} | n.d. | 28400^{b} | — | — |
| MCJ001-Dimer7-SS | 50 ± 3 | 29 ± 1 | 180 ± 20 | N > S > D | 6 |
| MCJ001-F—Ph—OH-RS/SR | 11900^{b} | 2660^{b} | 75200^{b} | N > S > D | 28 |
| MCJ001-F—Ph—OH-SS/RR | 1260^{b} | 514^{b} | 107^{b} | D > N > S | 12 |
| MCJ001-NA-Ph—OH-RS/SR | 75 ± 2 | 7620^{b} | 8900 ± 20 | S > N > D | 120 |
| MCJ001-NA-Ph—OH-SS/RR | 16 ± 2 | 1390^{b} | 5800 ± 400 | S > > N > D | 370 |
| MCJ002-Cl-RS | 7.4 ± 0.9 | 90 ± 10 | 390 ± 30 | S > N > D | 53 |
| MCJ002-Cl-SS | 8.3 ± 0.9 | 50 ± 8 | 270 ± 30 | S > N > D | 32 |
| MCJ002-F-RS | 7.0 ± 0.5 | 45 ± 3 | 280 ± 20 | S > N > D | 40 |
| Desipramine | 17.6 ± 0.7 | 0.83 ± 0.05 | 3190 ± 40 | N > S > D | |
| Duloxetine | 0.22 ± 0.06 | 4.2 ± 0.4 | 840 ± 90 | S > N > D | |
| Fluoxetine | 0.81 ± 0.02 | 240 ± 10 | 3600 ± 100 | S > N > D | |
| Imipramine | 1.4 ± 0.03 | 37 ± 2 | 8500 ± 100 | S > N > D | |
| Milnacipran | 9 ± 1 | 80 ± 10 | >10000 | S > N > D | |
| Nomifensine | 1010 ± 30 | 16.0 ± 0.4 | 56±3 | N > D > S | |
| Paroxetine | 0.13 ± 0.01 | 40 ± 2 | 490 ± 20 | S > N > D | |
| Reboxetine | 57.5 ± 0.6 | 7.2 ± 0.8 | >10000 | N > S > D | |
| Sertraline | 0.29 ± 0.01 | 420 ± 20 | 25 ± 2 | S > D > N | |
| Venlafaxine | 9.0 ± 0.3 | 1060 ± 40 | 9300 ± 50 | S > N > D | |

^{a}n ≧ 3 in all cases, except where indicated by “^{b}” where n = 1; “n.d.” = not determined.
Reference compounds (Tatsumi et al, Eur. J. Pharmacol., 340: 249-258 (1997) and E. Richelson, unpublished data—duloxetine, milnacipran, and reboxetine).

### Example 2

### Synthesis of (1R,2S)-1-(4-fluorophenyl)-3-(methylamino)-2-(naphthalen-2-yl)propan-1-ol (MCJ001-4FPh-OH-RS/SR; FIG. 1)

The compound MCJ001-4FPh-OH-RS/SR was made in four steps. Step 1: Preparation of 3-(4-fluorophenyl)-3-hydroxy-2-(naphthalen-2-yl)propanenitrile : LDA (250 mL, 92 0 mmol) solution was taken into an oven dried flask with stirrer at −78° C. and was left for 15 minutes for the temperature to equilibrate. A solution of cold 2-Naphthylacetonitrile (15.38 g, 92.0 mmol) in 100 mL of dry THF was added via syringe into the LDA solution at -78° C. slowly over 3 minutes. The solution turned bright orange, and was left for 30 minutes for the formation of anion. 4-Fluorobenzaldehyde (9.71 mL, 92.0 mmol) was then added drop-wise via syringe. The reaction mixture was left for 5 minutes and then checked for completion by TLC. The reaction was quenched with 20 mL 2:1 THF/Acetic acid solution. Cold bath was removed, and the reaction was allowed to reach room temperature slowly. The aqueous layer was extracted with EtOAc (3×25 mL), and the combined organic layers were washed with water and brine and dried over MgSO₄ Filtering and evaporating the volatiles under reduced pressure left a crude that was a mixture of the syn- and anti- diastereomeric 3-(4-fluorophenyl)-3-hydroxy-2-(naphthalen-2-yl) propanenitrile.

Step 2: Preparation of 3-amino-1-(4-fluorophenyl)-2- (naphthalen-2-yl) propan-1-ol: To a solution of diastereomeric 3- (4-fluorophenyl)-3-hydroxy-2-(naphthalen-2-yl)propanenitrile (4 g, 13.37 mmol) in 50 mL of dry THF, 4equivalents (55 mL, 54.9 mmol) of 1M Borane-THF solution was added via syringe under nitrogen. This mixture was stirred at 60° C. overnight and was quenched with cautious addition of NaHCO₃. The reaction was poured from the reaction vessel into a separatory funnel, and the aqueous layer was extracted with EtOAc (3×25 mL), and the combined organic layers were washed with water and brine and dried over MgSO₄. The combined organic layers were concentrated using a rotary evaporator to afford the product, which was subsequently converted to its hydrochloride salt using 1N HCl.

Yield of the mixture of syn- and anti-diastereomeric 3-amino-1-(4-fluorophenyl)-2-(naphthalen-2-yl)propan-1-ol was 96%; MS: m/z (ESI) 296=(M+1)⁻. Separation of these diastereomers proved to be difficult at this stage, hence this mixture was directly taken to the next step.

Step 3: Preparation of Tert-butyl-3- (4-fluorophenyl)-3-hydroxy - (naphthalen-2-yl)propylcarbamate: To a suspension of 3-amino-1-(4-fluorophenyl)-2-(naphthalen-2-yl)propan-1-ol (3.9 g ,13.20 mmol) in 50 mL of DCM, 3 equivalents (5.52 mL, 39.6 mmol) of Triethylamine was then added, followed by 1.25 equivalents (3.60 g, 16.51 mmol) of Boc anhydride. The reaction mixture was stirred at room temperature for about 1 hour. It was checked for completion of reaction by TLC and was then quenched with saturated NaHCO₃. Water was added, and the aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 40%EtOAc in hexanes) to afford the two diasteromeric products. Yield of the SS/RR diastereomer was 46.8% and that of the RS/SR diastereomer was 17.82%.

RS/SR: ¹H NMR (CDCl₃) δ 7.83-7.69(m, 3H), 7.62 (s, 1H), 7.50-7.42 (m, 2H), 7.19-7.09 (m, 3H), 6.94-6.84 (m, 2H), 5.02 (d, 1H, J=4.33Hz), 4.59 (br s, 1H), 3.87-3.69(m, 1H), 3.21-3.09 (m, 2H), 1.39 (s, 9H); MS: m/z (ESI) 396=(M+1)⁺ SS/RR: ¹H NMR (CDCl₃) δ 7.84-7.65 (m, 3H), 7.53-7.37 (m, 3H), 7.22-7.09 (m, 3H), 6.82 (t, 2H, J=8.67, 8.67 Hz), 4.98 (dd, 1H, J=8.48 Hz, j=3.01Hz), 4.73 (br s, 1H), 3.95-3.79(m, 1H), 3.71-3.51 (m, 1H), 3.28-3.17 (m, 1H). 1.43 (s, 9H); MS: m/z (ESI) 396=(M+1)⁺.

Step 4: Preparation of (1R, 2S)-1-(4-fluorophenyl)-3-(methylamino)-2-(naphthalen-2-yl)propan-1-ol: tert-butyl-(2S, 3R)-3-(4-fluorophenyl)-3-hydroxy-2-(naphthalen-2-yl)propylcarbamate (0.93 g, 2.352 mmol) was dissolved in 30 mL of dry THF, and 4 equivalents (0.357 g, 9.41 mmol) of LiAlH₄ was added to it and was refluxed (at 60° C.) under nitrogen overnight. The reaction was quenched by cautious addition of 10% NaOH dropwise. The aqueous layer was extracted with EtOAc (3 x 25 mL), and the combined organic layers were washed with water and brine 2-3 times and dried over MgSO₄. The organic layer was concentrated using a rotary evaporator and made into the hydrochloride salt with 1N HCl. The product was then dissolved in MeOH, and ether was added and allowed to crystallize at 4° C. White crystals were isolated and dried. Yield was 37%. ¹H NMR (CD₃OD) δ 7.93-7.79 (m, 3H), 7.67 (s, 1H), 7.56-7.48 (m, 2H), 7.42-7.12 (m, 3H), 7.02-6.91 (m, 2H), 5.19 (d, 1H, J=4.33 Hz), 3.74-3.41(m, 3H), 2.73 (s, 3H); MS: m/z (ESI) 310.0=(M+1)⁺.

### Example 3

### Synthesis of (1S,2S)-1-(4-fluorophenyl)-3-(methylamino)-2-(naphthalen-2-yl)propan-1-ol (MCJ001-4FPh-OH-SS/RR; FIG. 1)

The compound MCJ001-4FPh-OH-SS/RR was made as follows: 4-Fluorobenzaldehyde was reacted with a carbanion derived from β-acetonitrile as described above to give predominantly anti-adduct, which could be easily separated by recrystallization. The SS/RR diastereomer was obtained by recrystallizing the mixture of diastereomers in methanol and EtOAc. The nitrile function was reduced with borane. THF complex and the primary amine thus produced was converted to (1S, 2S)-1-(4-fluorophenyl)-3-(methylamino)-2-(naphthalen-2-yl)propan-1-ol (MCJ001-4FPh-OH-SS/RR; FIG. 1) by a two-step procedure, namely, conversion of the primary amine to t-butylcarbamate followed by reduction with borane-THF or lithum aluminum hydride (LAH). The final step yield was 72.3%; SS/RR: ¹H NMR (CD₃OD) δ 7.81-7.70 (m, 3H), 7.56 (s, 1H), 7.47-7.39 (m, 2H), 7.25-7.12 (m, 3H), 6.84 (t, 2H, J=8.85, 8.85 Hz), 5.04 (d, 1H, J=9.2 Hz), 3.97-3.82(m, 1H), 3.52-3.35 (m, 2H), 2.76 (s, 3H), MS: m/z (ESI) 310.34=(M+1)⁺.

### Example 4

### Synthesis of (1R, 2R/1S, 2S)-1-(4-bromophenyl)-3-(methylamino)-2-phenylpropan-1-ol (MCJ001-Br-Ph-OH-RS/SR; FIG. 2)

The compound MCJ001-Br-Ph-OH-RS/SR was made in four steps. Step 1: Preparation of 3-(4-bromophenyl)-3-hydroxy-2-phenylpropanenitrile: LDA (62.5 mL, 23 0 mmol) solution was taken into an oven dried flask with stirrer at −78° C. and was left for 15 minutes for the temperature to equilibrate. A solution of cold Phenylacetonitrile (2.65 mL, 23 0 mmol) in 25 mL of dry THF was added via syringe into the LDA solution at −78° C. slowly over 3 minutes. The solution turned bright orange, and was left for 30 minutes for the formation of anion. 2.69 mL of 4-Bromobenzaldehyde (2.69 mL, 23.0 mmol) was then added drop-wise via syringe. The reaction mixture was left for 5 minutes and then checked for completion by TLC. The reaction was quenched with 20 mL 2:1 THF/Acetic acid solution. Cold bath was removed, and the reaction was allowed to reach room temperature slowly. The aqueous layer was extracted with EtOAc (3×25 mL), and the combined organic layers were washed with water and brine and dried over MgSO₄. Filtering and evaporating the volatiles under reduced pressure left a solid that was a mixture of the syn- and anti- diastereomeric. The resulting residue was purified by flash chromatography over silica gel (elution with 15% to 30% EtOAc in hexanes) to remove the impurities. Yield was 34%.

Step 2: Preparation of 3-amino-1-(4-bromophenyl)-2-phenylpropan-1-ol: To a solution of diastereomeric 3-(4-bromophenyl)-3-hydroxy-2-phenylpropanenitrile (2.3 4 g, 7.74 mmol) in 25 mL of dry THF, 4 equivalents (31.0 mL, 31.0 mmol) of 1M Borane-THF solution was added via syringe under nitrogen. This mixture was stirred at 60° C. overnight and was quenched with cautious addition of NaHCO₃. The reaction was poured from the reaction vessel into a separatory funnel, and the aqueous layer was extracted with EtOAc (3×25 mL), and the combined organic layers were washed with water and brine and dried over MgSO₄. The combined organic layers were concentrated using a rotary evaporator to afford the product, which was subsequently converted to its hydrochloride salt using 1N HCl. Yield of the mixture of syn- and anti-diastereomeric 3-amino-1-(4-bromophenyl)-2-phenylpropan-1-ol was 97%. Separation of these diastereomers proved to be difficult at this stage; hence this mixture was directly taken to the next step.

Step 3: Preparation of tert-butyl 3-(4-bromophenyl)-3-hydroxy-2-phenylpropylcarbamate: To a suspension of diastereomeric 3-amino-1-(4-bromophenyl)-2-phenylpropan-1-ol (2.3 g, 7.51 mmol) in 50 mL of DCM, 3 equivalents (3.14 mL, 22.53 mmol) of Triethylamine was then added, followed by 1.25 equivalents (2.05 g, 9.39 mmol) of Boc anhydride. The reaction mixture was strirred at room temperature for about 1 hour. It was checked for completion of reaction by TLC and was then quenched with saturated NaHCO₃. Water was added, and the aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 40% EtOAc in hexanes) but the two diastereomers could not be separated. Yield of the mixture of diastereomers was 39%.

Step 4: Preparation of (1S,2S)-1-(4-bromophenyl)-3-(methylamino)-2-phenylpropan-1-ol and (1R,2S)-1-(4-bromophenyl)-3-(methylamino)-2-phenylpropan-1-ol: Tert-butylcarbamate was dissolved in 15 mL of dry THF and 4 equivalents of LiAlH₄ was added to them and were refluxed (at 60° C.) under nitrogen overnight. The reaction was quenched by cautious addition of 10% NaOH dropwise. The aqueous layer was extracted with EtOAc (3×25 mL), and the combined organic layers were washed with water and brine 2-3 times and dried over MgSO₄. The organic layer was concentrated using a rotary evaporator and made into the hydrochloride salt with 1N HCl. The product was dissolved in MeOH and was purified by reverse phase HPLC (Vydac column, C-18, 2.2×25 cm, elution with 10% B-100% B in 30 min; B=80% aq. CH₃CN with 0.1% TFA, A=H₂O with 0.1% TFA); FR 10 mL/min, λₘₐₓ=254 nm.). Two peaks were found; RT=17.671 min and 21.143 min. The solvents were evaporated off and corresponding HCl salts were made with 1N HCl. Yields were as follows: 13.34% for the SS/RR and 8.62% for the RS/SR.

SS/RR: ¹H NMR (CD₃OD) δ 7.36-7.26 (m, 4H), 7.19 (s, 1H), 7.17-7.09 (m, 3H), 7.07-7.02 (m, 1H), 5.01 (d, 1H, J=4.52 Hz), 3.58-3.33 (m, 3H), 2.66 (s, 3H); MS: m/z (ESI) 320.17=split peaks because of Br (M+1)⁺

RS/SR: ¹H NMR (CD₃OD) δ 7.34-7.26 (m, 3H), 7.26-7.21 (m, 3H), 7.19-7.08 (m, 3H), 5.01 (d, 1H, J=4.14 Hz), 3.52-3.36 (m, 3H), 2.66 (s, 3H); MS: m/z (ESI) 320.20=split peaks because of Br (M+1)⁺

Example 5

### Synthesis of (2S,2′S,3R,3′R)-3,3′-(heptane-1,7-diylbis(oxy))bis(N-methyl-2-(naphthalen-2-yl)-3-phenylpropan-1-amine) (MCJ001-Dimer?-SS; FIG. 3)

The compound MCJ001-Dimer7-SS was made in two steps. To a solution of tert-butyl-(2S,3S)-3-hydroxy-2-(naphthalen-2-yl)-3-phenylpropyl-(methyl)carbamate (Boc-PRC-200, 0.20 g, 0.511 mmol) in dry DMF (3 mL) was added NaH (0.098 g, 2.043 mmol) followed by 1,7-dibromoheptane (0.044 mL, 0.255 mmol) in dry DMF (1 mL) drop-wise at 0° C. (ice-bath) with stirring under nitrogen. The stirring was continued for 2 hours at room temperature. The reaction was quenched with water (2 mL) and extracted with diethyl ether (3×15 mL), the combined organic extracts were washed with brine, dried over MgSO₄, filtered and concentrated under reduced pressure. The crude was purified by silica-gel column chromatography using 20% ca/hex as eluent at afford a gummy solid (Boc-PRC200-heptyldimer), which was dissolved in 4N HCl-dioxane (5 mL) and stirred at room temperature under nitrogen for 45 minutes. The solvent was removed under reduced pressure, and the residue was washed with cold diethyl ether to afford the title compound as clear gummy solid (Yield 42% over two steps).

¹H NMR (MeOD) δ 7.72-7.77 (m, 6H), 7.60 (br s, 2H), 7.40-7.47 (m, 4H), 7.26 (dd, 2H, J=8.5, 1.6 Hz), 7.06-7.12 (m, 10H), 4.64 (d, 2H, J=8.8 Hz), 3.80 (dd, 2H, J=12.5, 5.9 Hz), 3.51 (m, 2H), 3.44 (m, 2H), 3.29-3.34 (m, 4H), 2.71 (br s, 6H), 1.56 (m, 4H), 1.33 (m, 6H). MS: m/z (ESI) 679.39 (M⁺1)⁺.

### Example 6

### Synthesis of (1R, 2R/1S, 2S))-1-(4-fluorophenyl)-3-(methylamino)-2-phenylpropan-1-ol (MCJ001-F-Ph-OH-RS/SR and SS/RR; FIG. 4)

The compounds MCJ001-FPh-OH-RS/SR and SS/RR were made in six steps. Step 1: Preparation of 3-(4-fluorophenyl)-3-hydroxy-2-phenylpropanenitrile: LDA (250 mL, 92.0 mmol) solution was taken into an oven dried flask with stirrer at −78° C. and was left for 15 minutes for the temperature to equilibrate. A solution of cold Phenylacetonitrile (10.62 mL, 92.0 mmol) in 100 mL of dry THF was added via syringe into the LDA solution at −78° C. slowly over 3 minutes. The solution turned bright orange, and was left for 30 minutes for the formation of anion. 4-Fluorobenzaldehyde (9.71 mL, 92.0 mmol) was then added drop-wise via syringe. The reaction mixture was left for 5 minutes and then checked for completion by TLC. The reaction was quenched with 20 mL 2:1 THF/Acetic acid solution. Cold bath was removed, and the reaction was allowed to reach room temperature slowly. The aqueous layer was extracted with EtOAc (3×25 mL), and the combined organic layers were washed with water and brine and dried over MgSO₄ Filtering and evaporating the volatiles under reduced pressure left a sticky solid that was a mixture of the syn- and anti- diastereomeric residue. The resulting residue was purified by flash chromatography over silica gel (elution with 15% to 40% EtOAc in hexanes) to remove the impurities. Yield was about 33%.

Step 2: Preparation of 3-amino-1-(4-fluorophenyl)-2-phenylpropan-1-ol: To a solution of diastereomeric 3-(4-fluorophenyl)-3-hydroxy-2-phenylpropanenitrile (7.0 g, 29.0 mmol) in 50 mL of dry THF, 4 equivalents (116 mL, 116.0 mmol) of 1M Borane-THF solution was added via syringe under nitrogen. This mixture was stirred at 60° C. overnight and was quenched with cautious addition of NaHCO₃. The reaction was poured from the reaction vessel into a separatory funnel, and the aqueous layer was extracted with EtOAc (3×25 mL), and the combined organic layers were washed with water and brine and dried over MgSO₄. The combined organic layers were concentrated using a rotary evaporator to afford the product which was subsequently converted to its hydrochloride salt using 1N HCl. Yield of the mixture of syn- and anti-diastereomeric 3-amino-1-(4-fluorophenyl)-2-(naphthalen-2-yl)propan-1-ol was 97.8%; MS: m/z (ESI) 246.1=(M+1)⁺. Separation of these diastereomers proved to be difficult at this stage; hence this mixture was directly taken to the next step. Step 3: Preparation of tert-butyl-3-(4-fluorophenyl)-3-hydroxy-2-phenylpropylcarbamate: To a suspension of diastereomeric 3-amino-1-(4-fluorophenyl)-2-phenylpropan-1-ol (7.0 g, 28 5 mmol) in 50 mL of DCM, 3 equivalents (11.93 mL, 86.0 mmol) of Triethylamine was then added, followed by 1.25 equivalents (7.79 g, 35.7 mmol) of Boc anhydride. The reaction mixture was stirred at room temperature for about 1 hour. It was checked for completion of reaction by TLC and was then quenched with saturated NaHCO₃. Water was added and the aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 40% EtOAc in hexanes) but they could not be separated at this stage. Yield of the mixture of diastereomers was 50.8%.

Step 4: Preparation of 1-(4-fluorophenyl)-3-(methylamino)-2-phenylpropan-1-ol: Tert-butyl-3-(4-fluorophenyl)-3-hydroxy-2-phenylpropylcarbamate (5g, 14.48 mmol) was dissolved in 150 mL of dry THF and 4 equivalents of LiAlH₄ (2.198 g, 57.9 mmol) was added to it and was refluxed (at 60° C.) under nitrogen overnight. The reaction was quenched by cautious addition of 10% NaOH drop-wise. The aqueous layer was extracted with EtOAc (3×25 mL), and the combined organic layers were washed with water and brine 2-3 times and dried over MgSO₄. The organic layer was concentrated using a rotary evaporator and made into the hydrochloride salt with 1N HCl. The product was then dissolved in MeOH, and ether was added and allowed to crystallize at 4° C. White crystals were isolated and dried. Yield was 96%. The diastereomers could not be separated at this stage even by HPLC. So another Boc reaction was done.

Step 5: Preparation of tert-butyl (2S,3S)-3-(4-fluorophenyl)-3-hydroxy-2-phenylpropyl(methyl)-carbamate: To a suspension of diastereomeric 1-(4-fluorophenyl)-3-(methylamino)-2-phenylpropan-1-ol (3.6 g, 13.88 mmol) in 30 mL of DCM, 3 equivalents (5.80 mL, 86.0 mmol) of Triethylamine was then added, followed by 1.25 equivalents (7.79 g, 41.6 mmol) of Boc anhydride. The reaction mixture was stirred at room temperature for about 1 hour. It was checked for completion of reaction by TLC and was then quenched with saturated NaHCO₃. Water was added, and the aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 25-30% EtOAc in hexanes) to afford the two pure diastereomers. Yield of the SS/RR was 43.1%, and the RS/SR was 39.3%. MS: m/z (ESI) 360.37 =(M+1)⁺ Step 6: The N-Boc protecting group of SS/RR and RS/SR tert-butyl 3-(4-fluorophenyl)-3-hydroxy-2-phenylpropyl(methyl)carbamate was readily removed by reacting it for 1 hour at room temperature with excess 2M HCl in dioxane. The reaction mixture was evaporated under reduced pressure, and the gummy crude after leaching several times with dry diethyl ether was dried under high vacuum. ¹H NMR, and MS indicated that the desired product was pure.

SS/RR: ¹H NMR (CD₃OD) δ 7.26-7.16 (m, 3H), 7.14-7.01 (m, 4H), 6.87 (t, 2H, J=8.67, 8.85 Hz), 4.89 (d, 1H, J=7.9 Hz), 3.83-3.73 (m, 1H), 3.35 (d, 1H, J=5.46 Hz), 3.23-3.13 (m, 1H)2.74(s, 3H); MS: m/z (ESI) 260.27=(M+1)⁺ RS/SR: ¹H NMR (CD₃OD) δ 7.32-7.24 (m, 3H), 7.16-7.01 (m, 4H), 6.92 (t, 2H, J=8.67, 8.85 Hz), 5.01 (d, 1H, J=4.52 Hz), 3.56-3.33 (m, 3H), 2.65(s, 3H); MS: m/z (ESI) 260.34=(M+1)⁺.

### Example 7

### Synthesis of (1R,2S/IS,2R)-3-(methylamino)-1-(naphthalen-2-yl)-2-phenylpropan-1-ol (MCJ001-NA-Ph-OH-RS/SR and SS/RR; FIG. 5)

The compounds MCJ001-NA-Ph-OH-RS/SR and SS/RR were made in six steps.

Step 1: Preparation of 4-hydroxy-4-(naphthalen-1-yl)-3-phenylbutanenitrile: LDA (62.5 mL, 23 0 mmol) solution was taken into an oven dried flask with stirrer at −78° C. and was left for 15 minutes for the temperature to equilibrate. A solution of cold Phenylacetonitrile (2.65 mL, 23.0 mmol) in 25 mL of dry THF was added via syringe into the LDA solution at −78° C. slowly over 3 minutes. The solution turned bright orange, and was left for 30 minutes for the formation of anion. 1-Naphthaldehyde (3.12 mL, 23.0 mmol) was then added drop-wise via syringe. The reaction mixture was left for 5 minutes and then checked for completion by TLC. The reaction was quenched with 10 mL 2:1 THF/Acetic acid solution. Cold bath was removed, and the reaction was allowed to reach room temperature slowly. The aqueous layer was extracted with EtOAc (3×25 mL), and the combined organic layers were washed with water and brine and dried over MgSO₄. Filtering and evaporating the volatiles under reduced pressure left a sticky solid that was a mixture of the syn- and anti-diastereomeric residue. The resulting residue was purified by flash chromatography over silica gel (elution with 15% to 40% EtOAc in hexanes) to remove the impurities. Yield was about 22%.

Step 2: Preparation of 3-amino-1-(naphthalen-1-yl)-2-phenylpropan-1-ol: To a solution of diastereomeric 4-hydroxy-4-(naphthalen-1-yl)-3-phenylbutanenitrile (1.45 g, 5.05 mmol) in 25 mL of dry THF, (4equivalents (20.18 mL, 20.18 mmol) of 1M Borane-THF solution was added via syringe under nitrogen. This mixture was stirred at 60° C. overnight and was quenched with cautious addition of NaHCO₃. The reaction was poured from the reaction vessel into a separatory funnel, and the aqueous layer was extracted with EtOAc (3×25 mL), and the combined organic layers were washed with water and brine and dried over MgSO₄. The combined organic layers were concentrated using a rotary evaporator to afford the product which was subsequently converted to its hydrochloride salt using 1N HCl. Yield of the mixture of syn- and anti-diastereomeric 3-amino-1-(4-fluorophenyl)-2-(naphthalen-2-yl)propan-1-ol was 99%. Separation of these diastereomers proved to be difficult at this stage; hence this mixture was directly taken to the next step.

Step 3: Preparation of ethyl-3-hydroxy-3-(naphthalen-1-yl)-2-phenylpropylcarbamate: To a suspension of diastereomeric 3-amino-1-(naphthalen-1-yl)-2-phenylpropan-1-ol (1.45 g, 5.05 mmol) in 25 mL of DCM, 1.1 equivalents (0.531 mL, 5.55 mmol) of ethyl chloroformate was then added, followed by 1.6 equivalents (0.653 mL, 8.08 mmol) of dry pyridine at 0° C. The reaction mixture was stirred at 0° C. for about 10 minutes and then was allowed to stir at room temperature overnight. It was checked for completion of reaction by TLC and was diluted with DCM and poured into a separatory funnel containing 2N HCl (5 mL/g of the starting material). The aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 15-25% EtOAc in hexanes) to yield a gummy transparent product. Yield of the mixture of diastereomers was 53%.

Step 4: Preparation of 3-(methylamino)-1-(naphthalen-1-yl)-2-phenylpropan-1-ol: Ethyl-3-hydroxy-3-(naphthalen-1-yl)-2-phenylpropylcarbamate (5 g, 14.48 mmol) was dissolved in 150 mL of dry THF and 4 equivalents (2.198 g, 57.9 mmol) of LiAlH₄ was added to it and was refluxed (at 60° C.) under nitrogen overnight. The reaction was quenched by cautious addition of 10% NaOH drop-wise. The aqueous layer was extracted with EtOAc (3×25 mL), and the combined organic layers were washed with water and brine 2-3 times and dried over MgSO₄. The organic layer was concentrated using a rotary evaporator and made into the hydrochloride salt with 1N HCl to yield a sticky solid. The diastereomers could not be separated at this stage even by HPLC. So another Boc reaction was performed.

Step 5: Preparation of tert-butyl 3-hydroxy-3-(naphthalen-1-yl)-2-phenylpropyl(methyl)carbamate: To a suspension of diastereomeric 3-(methylamino)-1-(naphthalen-1-yl)-2-phenylpropan-1-ol (0.9 g, 3.24 mmol) in 15 mL of DCM, 3 equivalents (1.37 mL, 9.73 mmol) of Triethylamine was then added, followed by 1.25 equivalents (0.885 g, 4.06 mmol) of Boc anhydride. The reaction mixture was stirred at room temperature for about 45 minutes. It was checked for completion of reaction by TLC and was then quenched with saturated NaHCO₃. Water was added, and the aqueous layer was extracted with DCM (3×25 mL), and the combined organic layers were washed with water and dried over MgSO₄. The combined organic layers were concentrated using a rotary evaporator. The resulting residue was purified by flash chromatography over silica gel (elution with 20-30% EtOAc in hexanes) to afford the two pure diastereomers. Yield of the SS/RR was 43.1% (white solid), and the RS/SR was 39.3% (colorless sticky solid).

Step 6: The N-Boc protecting group of SS/RR and RS/SR tert-butyl 3-hydroxy-3-(naphthalen-1-yl)-2-phenylpropyl(methyl)carbamate was readily removed by reacting it for 1 hour at room temperature with excess 2M HCl in dioxane. The reaction mixture was evaporated under reduced pressure, and the gummy crude after leaching several times with dry diethyl ether was dried under high vacuum. ¹H NMR, and MS indicated that the desired product was pure.

SS/RR: ¹H NMR (CD₃OD) δ 8.19 (br s, 1H), 7.89-7.68 (m, 3H), 7.59-7.31 (m, 5H), 7.12 (s, 3H), 5.67 (d, 1H, J=8.38 Hz), 3.90-3.74 (m, 1H), 3.71-3.58 (m, 1H), 3.57-3.42 (m, 1H), 2.75 (s, 3H); MS (ESI): m/z 292.07=(M+1)⁺.

RS/SR: ¹H NMR ¹H NMR (CD₃OD) δ 8.14-8.07 (m, 1H), 7.93-7.86 (m, 1H), 7.78-7.71 (m, 1H),7.55-7.47 (m, 2H), 7.31-7.16 (m, 5H), 7.08-6.99 (m, 2H), 5.89 (d, 1H, J=3.77 Hz), 3.78-3.49 (m, 3H), 2.75 (s, 3H); MS (ESI): m/z 292.07=(M+1)⁺.

### Example 8

### Synthesis of (2S,3R)-3-chloro-N,N-dimethyl-2-(naphthalen-2-yl)-3-phenylpropan-1-amine (MCJ002-Cl-RS, FIG. 6)

The compound MCJ002-Cl-RS was made in one step. Step 1: Preparation of (2S, 3R)-3-chloro-N,N-dimethyl-2-(naphthalen-2-yl)-3-phenylpropan-1-amine: 3 mL of dry DCM was added to (0.100 g, 0.426 mmol) of (1S, 2S)-3-(dimethylamino)-2-(naphthalen-2-yl)-1-phenylpropan-1-ol(PRC-184), followed by 1 mL of thionyl chloride. The reaction mixture was stirred at room temperature for 30 minutes under nitrogen, after which the organic layers were concentrated using a rotary evaporator. NaHCO₃ was added and the aqueous layer was extracted with DCM (3×25 mL), and the combined organic layers were washed with water and dried over MgSO₄. The resulting residue (free amine) was purified by flash chromatography over silica gel (elution with 1% MeOH/DCM) to afford the product. The solvents were evaporated off using a rotary evaporator, and the hydrochloride salt was made with 2N HCl, which was an off white powder. Yield was 92%. ¹H NMR (CD₃OD) δ 7.80-7.69 (m,4H), 7.49-7.41(m, 2H), 7.36-7.29 (m, 2H), 7.27-7.20(m, 2H), 7.15-7.06 (m, 3H), 5.31 (d, J=8.85 Hz, 1H), 4.17-3.98 (m, 3H), 2.94(s, 3H), 2.87(s, 3H); MS: m/z (ESI) 324.27=(M+1)⁺.

### Example 9

### Synthesis of (2S,3S)-3-chloro-N,N-dimethyl-2-(naphthalen-2-yl)-3-phenylpropan-1-amine (MCJ002-C1-SS; FIG. 6) The compound MCJ002-C1-SS was made in one step.

Step 1: Preparation of (2S,3S)-3-chloro-N,N-dimethyl-2-(naphthalen-2-yl)-3-phenylpropan-1-amine: 2 mL of dry DCM was added to (0.055 g, 0.180 mmol) of (1R,2,5)-3-(dimethylamino)-2-(naphthalen-2-yl)-1-phenylpropan-1-ol, followed by 1 mL of thionyl chloride. The reaction mixture was stirred at room temperature for 30 minutes under nitrogen, after which the organic layers were concentrated using a rotary evaporator. NaHCO₃ was added and the aqueous layer was extracted with DCM (3×25 mL), and the combined organic layers were washed with water and dried over MgSO₄. The resulting residue (free amine) was purified by flash chromatography over silica gel (elution with 1% MeOH/DCM) to afford the product. The solvents were evaporated off using a rotary evaporator, and the hydrochloride salt was made with 2N HCl, which was a white powder. Yield was 60.2%. ¹H NMR (CD₃OD) δ 7.97-7.83 (m, 4H), 7.58-7.43(m, 3H), 7.34 (s, 5H), 5.38 (d, J=6.8 Hz, 1H), 4.07-3.89 (m, 3H), 2.83(s, 3H), 2.76 (s, 3H); MS: m/z (ESI) 324.36=(M+1)⁺.

### Example 10

### Synthesis of (2S,3R)-3-fluoro-N,N-dimethyl-2-(naphthalen-2-yl)-3-phenylpropan-1-amine (MCJ002-F-RS; FIG. 7)

The compound MCJ002-F-RS was made in one step. Step 1: Preparation of (2S, 3R)-3-fluoro-N,N-dimethyl-2-(naphthalen-2-yl)-3-phenylpropan-1-amine. To 0.140 g of (1S,2S)-3-(dimethylamino)-2-(naphthalen-2-yl)-1-phenylpropan-1-ol (PRC-184), 5 mL of 2N NaOH was added and stirred, and free amine was extracted with DCM and dried. 3 mL of dry DCM was added to the free amine (0.060 g, 0.196 mmol), followed by 4 equivalents (0.103 mL, 0.786mmol) of DAST was added very carefully under nitrogen and stirred at room temperature overnight. The reaction was quenched with NaHCO₃ very carefully (on ice) and stirred at room temperature for an hour. The aqueous layer was extracted with EA (3×25 mL), and the combined organic layers were washed with water and brine and dried over MgSO₄. The organic layers were concentrated using a rotary evaporator. The resulting residue was purified by flash chromatography over silica gel (elution with 2% MeOH/DCM) to afford the product. The solvents were evaporated off using a rotary evaporator and HCl salt was made with 2N HCl which was a light brown solid. Yield was 71.2%. ¹H NMR (DMSO) δ 9.36 (br s, 1H), 7.97-7.69 (m, 4H), 7.59-7.39 (m, 3H), 7.34-7.12 (m, 5H), 5.87 (dd, 1H, J=46.7, 9.05 Hz), 4.21-4.03 (m, 1H), 3.98-3.71 (m, 2H), 2.80 (d, 3H, J=4.7 Hz), 2.72 (d, 3H, J=4.33 Hz); MS: m/z (ESI) 308.36=(M+1)⁺.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising 1-(4-fluorophenyl)-3-(methylamino)-2-(naphthalen-2-yl)propan-1-ol or a salt thereof

2. The composition of claim 1, wherein said 1-(4-fluorophenyl)-3-(methylamino)-2-(naphthalen-2-yl)propan-1-ol comprises (1R, 2R)-1-(4-fluorophenyl)-3-(methylamino)-2-(naphthalen-2-yl)propan-1-ol or (1S, 2S)-1-(4-fluorophenyl)-3-(methylamino)-2-(naphthalen-2-yl)propan-1-ol.

3. The composition of claim 1, wherein said 1-(4-fluorophenyl)-3-(methylamino)-2-(naphthalen-2-yl)propan-1-ol comprises (1R, 2S)-1-(4-fluorophenyl)-3-(methylamino)-2-(naphthalen-2-yl)propan-1-ol or (1S, 2R)-1-(4-fluorophenyl)-3-(methylamino)-2-(naphthalen-2-yl)propan-1-ol.

4. The composition of claim 1, wherein said 1-(4-fluorophenyl)-3-(methylamino)-2-(naphthalen-2-yl)propan-1-ol comprises:
(i) two compounds selected from the group consisting of (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2S)-1-(4-fluorophenyl)-3-(methylamino)-2-(naphthalen-2-yl)propan-1-ol, and (1S, 2R)-1-(4-fluorophenyl)-3-(methylamino)-2-(naphthalen-2-yl)propan-1-ol; or
(ii) three compounds selected from the group consisting of (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2S)-1-(4-fluorophenyl)-3-(methylamino)-2-(naphthalen)-2-yl)propan-1-ol, and (1S, 2R)-1-(4-fluorophenyl)-3-(methylamino)-2-(naphthalen-2-yl)propan-1-ol; or
(iii) (1R, 2R)-1-(4-fluorophenyl)-3-(methylamino)-2-(naphthalen-2-yl)propan-1-ol, (1S, 2S)-1-(4-fluorophenyl)-3-(methylamino)-2-(naphthalen-2-yl)propan-1-ol, (1R, 2S)-1-(4-fluorophenyl)-3-(methylamino)-2-(naphthalen-2-yl)propan-1-ol, and (1S, 2R)-1-(4-fluorophenyl)-3-(methylamino)-2-(naphthalen-2-yl)propan-1-ol.

5. The composition of claim 1, wherein said composition comprises a pharmaceutically acceptable carrier.

6. A composition comprising 1-(4-bromophenyl)-3-(methylamino)-2-phenylpropan-1-ol or a salt thereof

7. The composition of claim 6, wherein said 1-(4-bromophenyl)-3-(methylamino)-2-phenylpropan-1-ol comprises (1R, 2R)-1-(4-bromophenyl)-3-(methylamino)-2-phenylpropan-1-ol or (1S, 2S)-1-(4-bromophenyl)-3-(methylamino)-2-phenylpropan-1-ol.

8. The composition of claim 6, wherein said 1-(4-bromophenyl)-3-(methylamino)-2-phenylpropan-1-ol comprises (1R, 2S)-1-(4-bromophenyl)-3-(methylamino)-2-phenylpropan-1-ol or (1S, 2R)-1-(4-bromophenyl)-3-(methylamino)-2-phenylpropan-1-ol.

9. The composition of claim 6, wherein said 1-(4-bromophenyl)-3-(methylamino)-2-phenylpropan-1-ol comprises:
(i) two compounds selected from the group consisting of (1R, 2R)-1-(4-bromophenyl)-3-(methylamino)-2-phenylpropan-1-ol, (1S, 2S)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol; or
(ii) three compounds selected from the group consisting of (1R, 2R)-1-(4-bromophenyl)-3-(methylamino)-2-phenylpropan-1-ol, (1S, 2S)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol; or
(iii) (1R, 2R)-1-(4-bromophenyl)-3-(methylamino)-2-phenylpropan-1-ol, (15, 25)-1-(4-bromophenyl)-3-(methylamino)-2-phenylpropan-1-ol, (1R, 2S)-1-(4-bromophenyl)-3-(methylamino)-2-phenylpropan-1-ol, and (1S, 2R)-1-(4-bromophenyl)-3-(methylamino)-2-phenylpropan-1-ol.

10. The composition of claim 6, wherein said composition comprises a pharmaceutically acceptable carrier.

11. A composition comprising 3,3′-(heptane-1,7-diylbis(oxy))bis(N-methyl-2-(naphthalen-2-yl)-3-phenylpropan-1-amine) or a salt thereof.

12. The composition of claim 11, wherein said 3,3′-(heptane-1,7-diylbis(oxy))bis(N-methyl-2-(naphthalen-2-yl)-3-phenylpropan-1-amine) comprises (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine).

13. The composition of claim 11, wherein said 3,3′-(heptane-1,7-diylbis(oxy))bis(N-methyl-2-(naphthalen-2-yl)-3-phenylpropan-1-amine) comprises (2S, 2′S, 3R, 3′R)-3,3′-(heptane-1,7-diylbis(oxy))bis(N-methyl-2-(naphthalen-2-yl)-3-phenylpropan-1-amine) or (2R, 2′R, 3S, 3′S)-3,3′-(heptane-1,7-diylbis(oxy))bis(N-methyl-2-(naphthalen-2-yl)-3-phenylpropan-1-amine).

14. The composition of claim 11, wherein said 3-azido-N-methyl-2-(naphthalen-2-yl)-3-phenylpropan-1-amine comprises:
(i) two compounds selected from the group consisting of (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′ S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine); or
(ii) three compounds selected from the group consisting of (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine); or
(iii) (2R, 2′R, 3R, 3′R)-3,3′-(heptane-1,7-diylbis(oxy))bis(N-methyl-2-(naphthalen-2-yl)-3-phenylpropan-1-amine) or (2S, 2′S, 3S, 3′S)-3,3′-(heptane-1,7-diylbis(oxy))bis(N-methyl-2-(naphthalen-2-yl)-3-phenylpropan-1-amine), (2S, 2′S, 3R, 3′R)-3,3′-(heptane-1,7-diylbis(oxy))bis(N-methyl-2-(naphthalen-2-yl)-3-phenylpropan-1-amine), and (2R, 2′R, 3S, 3′S)-3,3′-(heptane-1,7-diylbis(oxy))bis(N-methyl-2-(naphthalen-2-yl)-3-phenylpropan-1-amine).

15. The composition of claim 11, wherein said composition comprises a pharmaceutically acceptable carrier.

16-41. (canceled)
